(19) United States
(12) Patent Application Publication
(10) Pub. No.: US 2012/0004289 A1
(43) Pub. Date: Jan. 5, 2012

(54) ANNEXIN A11 AND ASSOCIATED GENES AS BIOMARKERS FOR CANCER

(75) Inventors: Jin Song, Clarksville, MD (US); Zhen Zhang, Dayton, MD (US)

(73) Assignee: The Johns Hopkins University, Baltimore, MD (US)

(21) Appl. No.: 13/255,015

(22) PCT Filed: Mar. 5, 2010
(86) PCT No.: PCT/US2010/026343

§ 371 Date: Sep. 6, 2011

Related U.S. Application Data
(60) Provisional application No. 61/158,043, filed on Mar. 6, 2009.

(51) Int. Cl. A61K 31/7088; C40B 30/02; A61P 35/00; C07K 16/24; C07K 16/18; G01N 33/53; C40B 30/04

(52) U.S. Cl. 514/44 R; 435/7.92; 436/501; 506/8; 506/9; 530/389.7; 530/389.2

(57) ABSTRACT

The instant invention provides methods and compositions for the diagnosis and treatment of cancer. The invention also provides method and compositions for determining if a subject is, or is at risk of becoming, chemoresistant.

### RELATED APPLICATIONS

This application claims the benefit of U.S. Provisional Application No. 61/158,043, filed Mar. 6, 2009. The entire contents of the aforementioned provisional application are hereby incorporated by reference.

### GOVERNMENT SUPPORT

The following invention was supported at least in part by Department of Defense IDEA grant DAMD17-OC03-IDEA, and the National Institutes of Health/NCI Grant No.; CA115102-01. Accordingly, the government has certain rights in the invention.

### BACKGROUND OF THE INVENTION

Ovarian cancer is the fifth leading cause of cancer death among U.S. women and has the highest mortality rate of all gynecologic cancers (1). Due to lack of effective screening tools and therapy, the mortality of ovarian cancer has not declined in the past two decades. Most cases of ovarian cancer, approximately 75%, are diagnosed at an advanced stage of the disease (1). While patients with early stage disease will have over a 74% chance of survival, those with advanced stage cancer will have overall survival rates of only 19-30% (1, 2). Administration of adjuvant chemotherapy consisting of a platinum compound (cisplatin or carboplatin) and a taxene remains the standard treatment for advanced stage cancer following an optimal primary debulking surgery (3). One of the most important clinical problems in the treatment of ovarian cancer is the intrinsic/acquired resistance to cisplatin-based chemotherapy. Although they are initially very responsive (80%) to cisplatin-based chemotherapy, 75% of patients easily develop cisplatin resistance and relapse within 2 years of primary therapy (4). The progression of cisplatin-resistant cancer confers poor prognosis and decreases overall survival of this disease.

Several mechanisms such as decreased drug accumulation, enhanced detoxification, drug sequestration, faster repair of cisplatin-DNA adducts and modulation of apoptotic pathways have been implicated in cisplatin resistance, but they are not sufficient to exhaustively explain this resistance emergence (5-9). Identification and characterization of more determinants of cisplatin resistance will advance our understanding of the varied mechanism that can contribute to this clinically relevant phenomenon, and lead to the development of new protein markers or to the establishment of new therapeutic strategies.

Accordingly, a need exists to better understand the molecular mechanism of chemoresistance in ovarian cancer subjects.

### SUMMARY OF THE INVENTION

The instant invention is based, at least in part, on work by the present inventors that has shown that knockdown of annexin A11 expression reduced cell proliferation and colony formation ability of ovarian cancer cells. The present inventors found that epigenetic silencing of annexin A11 conferred cisplatin resistance to ovarian cancer cells. Through a comprehensive time course study of cisplatin response in ovarian cancer cells with/without suppression of annexin A11 expression using whole-genome oligonucleotide microarrays, the present inventors have identified a set of differentially expressed genes associated with annexin A11 expression and some patterns of gene expressions in response to cisplatin exposure.

Accordingly, in one aspect, the instant invention provides a method of determining if a subject has become or is at risk of becoming chemoresistant, comprising obtaining a biological sample from the subject, and measuring the level of one or more proteins selected from the group consisting of: PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, and ARF3, wherein an increased level of one or more proteins is indicative that the subject is or will become chemoresistant.

In one embodiment, PLEKHM1, KRTAP3-1, MB2, DERP12, and ZA31P are increased at least 8 hours after a subject is treated with chemotherapy.

In another embodiment, PLEKHM1, A_{—}24_P932355, PCSK9, MB2, and ZA31P are increased at least 16 hours after a subject is treated with chemotherapy.

In another embodiment, PLEKHM1, A_{—}24_P932355, MB2, ZA31P, DERP12, RC1, JAK3, BC038245, HSPA2, SOST, METTL7A, NGEF, GPR30, GLRX, A_{—}23_P72014, L3MBTL, KCNMB4, GNAZ, PCSK9, AK096109, COL9A3, and ARF3 are increased at least 24 hours after a subject is treated with chemotherapy.

In one embodiment of any one of the above aspects, annexin A11 gene expression is also decreased.

In another aspect, the present invention features a method of determining if a subject has become or is at risk of becoming chemoresistant, comprising obtaining a biological sample from the subject, and measuring the level of one or more proteins selected from the group consisting of H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, and NAV3, wherein a decreased level of one or more proteins is indicative that the subject is or will become chemoresistant.

In one embodiment, H1F0 and PLEKHM1 are decreased at least 8 hours after a subject is treated with chemotherapy.

In another embodiment, SERPINB2, MX1, KRT6C, ISGF3G, IFI44, IFIT1, IFI44L, ADAMTS1, pTR7, DHRS2, IL-8, CXCL2, MIRH1, PLEKHM1, A_{—}24_P932355 and IL1R2 are decreased at least 16 hours after a subject is treated with chemotherapy.

In another embodiment, PLEKHM1, H1F0, PLEKHM1, SERPINB2, MX1, KRT6C, ISGF3G, IFI44, IFIT1, IFI44L, ADAMTS1, pTR7, IL-8, CXCL2, MIRH1, IL1R2, ZCCHC2, UBE2E1, ZNF358, H1F0, KRT6C, KRT6A, ISGF3G, IFI44, MX1, IFIT1, IFI44L, MX2, FLJ20035, ATP8A2, pTR7, TNC, DHRS2, SNHG7, ILIR2, IL8, CXCL2, A_{—}24_P707102, SERPINB2, NAV3, A_{—}24_P932355 and ADAMTS1 are decreased at least 24 hours after a subject is treated with chemotherapy.

In one embodiment of any one of the above aspects, annexin A11 gene expression is also decreased.

In another further embodiment, PLEKHM1 and A_{—}24_P932355 are decreased when annexin A11 gene expression is also decreased.

In another aspect, the present invention features a method of determining if a subject has become or is at risk of becoming chemoresistant, comprising obtaining a biological sample from the subject, and measuring the level of one or more proteins selected from the group consisting of: HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, and PDZD2, wherein an increased level of the protein is indicative that the subject is or will become chemoresistant.

In another particular aspect, the present invention features a method of determining if a subject has become or is at risk of becoming chemoresistant, comprising obtaining a biological sample from the subject, and measuring the level of one or more proteins selected from the group consisting of: HIST1H2BM, LOC391566, EIF4EBP2, HISTH2BK, SCML1, and ANXA11, wherein a decreased level of the protein is indicative that the subject is or will become chemoresistant.

In one embodiment of the above aspects, the increased or decreased level of the protein is associated with annexin A11 gene expression.

In another embodiment, the one or more proteins is selected from the group consisting of: HMOX and LY6D.

In another embodiment, the protein is HMOX1.

In another embodiment of any one of the above aspects, the subject is chemoresistant to a platinum based chemotherapeutic.

In another further embodiment, the platinum based therapeutic is selected from Carboplatin, Cisplatin, Oxaliplatin, BBR3464, and Satraplatin. In a related embodiment, the platinum based therapeutic is cisplatin.

In another embodiment of any one of the above aspects, the subject has a cell proliferative disorder.

In another further embodiment, the cell proliferative disorder is cancer. In a related embodiment, the cancer is selected from pancreatic, kidney, stomach, colon, lung, bladder, prostate, uterine, breast or ovarian cancer. In another further embodiment, the cancer is ovarian cancer.

In another embodiment of any one of the above aspects, the increase or decrease of the level of the protein is relative to a control.

In another further embodiment, the control is a sample of a non-cancerous tissue. In a related embodiment, the control is a sample from a subject that expresses annexin A11.

In another aspect, the present invention features a method of determining if a subject having ovarian cancer has become, or is at risk of becoming chemoresistant, comprising obtaining a biological sample from the subject; and measuring the level of one or more proteins selected from the group consisting of H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11 polypeptide in the sample, wherein a decreased level of one or more proteins is indicative that the subject is or will become chemoresistant.

In another aspect, the present invention features a method of determining if a subject having ovarian cancer has become, or is at risk of becoming chemoresistant, comprising obtaining a biological sample from the subject; and measuring the level of one or more proteins selected from the group consisting of: PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, and PDZD2 polypeptide in the sample, wherein an increased level of one or more proteins is indicative that the subject is or will become chemoresistant.

In one embodiment of the above aspects, the one or more proteins are measured 8, 16 or 24 hours after treatment with a chemotherapeutic.

In another embodiment of the above aspects, the subject is chemoresistant to a platinum based chemotherapeutic.

In a further embodiment, the platinum based therapeutic is selected from Carboplatin, Cisplatin, Oxaliplatin, BBR3464, and Satraplatin. In a related embodiment, the platinum based therapeutic is cisplatin.

In one embodiment of the above aspects, the decrease in the level of the one or more proteins is relative to a control.

In a further embodiment, the control is a sample of a non-cancerous tissue.

In another embodiment, the control is a sample from a subject that expresses annexin A11.

In another aspect, the present invention features a method of determining if subject is likely to have a recurrence of cancer comprising obtaining a biological sample from the subject, and measuring the level of one or more proteins selected from the group consisting of H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11 polypeptide in the sample, wherein a decreased level of one or more proteins is indicative that the subject will have a recurrence of cancer.

In another aspect, the present invention features a method of determining if a subject is likely to have a recurrence of cancer comprising obtaining a biological sample from the subject, and measuring the level of one or more proteins selected from the group consisting of: PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, and PDZD2 polypeptide in the sample, wherein an increased level of one or more proteins is indicative that the subject will have a recurrence of cancer.

In still another aspect, the present invention features a method of treating a subject having cancer comprising administering to the subject a nucleic acid molecule encoding one or more proteins selected from the group consisting of: PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, PDZD2, H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11, wherein the nucleic acid molecule is capable of producing the one or more polypeptides in the cells of the subject.

In one embodiment of the above aspects, the one or more proteins is selected from the group consisting of: HMOX and LY6D.

In one embodiment of the above aspects, the one or more proteins is HMOX.

In another embodiment, the nucleic acid molecule is a nucleic acid vector.

In a further embodiment, the vector is a viral vector.

In still another embodiment, the nucleic acid molecule is administered with one or more chemotherapeutic molecules.

In another aspect, the present invention features a method of determining the prognosis of a subject having cancer comprising obtaining a biological sample from the subject, and measuring the level of one or more proteins selected from the group consisting of H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11 polypeptide in the sample, wherein a decreased level of one or more proteins is indicative of a poor prognosis.

In another aspect, the present invention features a method of determining the prognosis of a subject having cancer comprising obtaining a biological sample from the subject; and measuring the level of one or more proteins selected from the group consisting of: PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, and PDZD2 polypeptide in the sample, wherein an increased level of one or more proteins is indicative of a poor prognosis.

In one embodiment of the above aspects, the one or more proteins is selected from HMOX and LY6D.

In another embodiment, the one or more proteins is HMOX.

In one embodiment of the above aspects, the subject is chemoresistant to a platinum based chemotherapeutic.

In another embodiment, the platinum based therapeutic is selected from Carboplatin, Cisplatin, Oxaliplatin, BBR3464, and Satraplatin.

In a further embodiment, the platinum based therapeutic is cisplatin.

In one embodiment of the above aspects, the cancer selected from pancreatic, kidney, stomach, colon, lung, bladder, prostate, uterine, breast and ovarian cancer.

In a further embodiment, the cancer is ovarian cancer.

In one embodiment of the above aspects, the increase or decrease of the level of the protein is relative to a control.

In one embodiment of the above aspects, the control is a sample of a non-cancerous tissue.

In one embodiment, the control is a sample from a subject that expresses annexin A11.

In one embodiment of the above aspects, the one or more proteins are measured 8, 16 or 24 hours after treatment with a chemotherapeutic.

In another aspect, the present invention features a kit for the diagnosis of cancer comprising an antibody that specifically binds to one or more proteins selected from PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, PDZD2, H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11, and instructions for use.

In still another aspect, the present invention features a kit for determining the prognosis of a subject having cancer comprising an antibody that specifically binds to one or more proteins selected from PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, PDZD2, H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11, and instructions for use.

In one embodiment of the above aspects, the detection of an increased or decreased level of antibodies relative to a control is indicative of cancer.

In another embodiment, the cancer is ovarian cancer.

### DESCRIPTION OF THE DRAWINGS

FIG. 1A-D shows knockdown of annexin A11 expression in ovarian cancer cells. (A and B) Effect of silencing of annexin A11 using different siRNA. 2008 cells were treated with one (A1 or A2 or A3) or a combination (A1-3) of three stealth RNA against annexin A11 or nonspecific sequence (-Ctr) at the concentration of 40 nM or without treatment (Wt) for 3 days. Immunoblot analysis (A) and real-time PCR (B) were performed to confirm the suppression of annexin A11 mRNA and protein expressions in the cells. β-Actin was taken as an additional control for equal sampling in immunoblot analysis (A). The relative mRNA expression level of each sample was normalized to GAPDH expression and compared with -Ctr sample. *P<0.05 (B). (C) Dose-dependent silencing of annexin A11 by siRNA. 2008 cells were treated with RNAi (A1) at the indicated concentrations of 40, 20, 10, or 5 nM or -Ctr at the concentration of 40 nM or without treatment (Wt) for 3 days. Immunoblot analysis (C) was performed to check the annexin A11 expression levels in the cells. β-Actin was taken as an additional control for equal sampling. (D) Duration of silencing of annexin A11 using siRNA. 2008 or HEY cells were treated with RNAi (A1) or -Ctr at the concentration of 40 nM. Immunoblot analysis was performed to analyze the annexin A11 expression levels in these cells. Note that the level of annexin A11 protein was significantly decreased by day 3 (2008; left top), day 10 (2008; left bottom), day 2 (HEY; right top), or day 7 (HEY; right bottom), respectively. β-Actin or annexin A5 was taken as a loading control or off-target effect control.

FIG. 2 A-D shows epigenetic silencing of annexin A11 reduces cell proliferation, colony formation ability, and confers cisplatin resistance to ovarian cancer cells. (A and B) Cell proliferation assay. 2008 (A) or HEY (B) cells were treated with RNAi (A1) or -Ctr at the concentration of 40 nM for 3 days, respectively, and then plated at 3000 viable cells per well into 96-well plates. Every 24 hours, one plate was subjected to assay by CCK-8 kit. The data in each time point are averaged values from eight replicates (P<0.05). (C) Colony formation assay. 2008 cells were treated in the same way as above for 3 days and then plated at 3000 viable cells per well into six-well plates. Six days after plating, cells were fixed with methanol and stained with 0.1% crystal violet and colonies were counted. The experiment was performed in six replicates (P<0.01). (D) Cell cytotoxicity assay. 2008 cells were treated in same way as above for 3 days and then plated at 3000 viable cells per well into 96-well plates. After incubating overnight, cells were treated with various concentration of cisplatin diluted in 100 μl of conditioned medium (the final concentrations of cisplatin were 0, 1.56, 3.13, 6.25, 12.5, 25, 50, and 100 μg/ml). After continuous incubation for 72 hours, the plates were subjected to assay by CCK-8 kit. The experiment was performed in three replicates (P<0.01). Three independent experiments were performed for each assay.

FIG. 3 A-D shows dynamic response of gene expression to cisplatin treatment and annexin A11-associated gene expression alterations. (A-C) Hierarchical clustering of gene expression alterations. Hierarchical clustering of genes either upregulated or downregulated more than two-fold change at 8 (A), 16 (B), and 24 hours (C) compared with 0 hour in both RNAi (R groups) and control (N groups) cell lines are shown. (D) Hierarchical clustering of genes with a fold up-regulation or down-regulation of at least two (R vs N) at every single time point are also shown. R1-R4 or N1-N4 represents different time points at 0, 8, 16, or 24 hours in order, respectively, in R or N groups. Clustering was performed using the Cluster and TreeView software. Genes that were increased are shown in red, whereas genes that were decreased are indicated in blue.

FIG. 4 A-C shows validation of DNA microarray data and immunohistochemical analysis. (A) Validation of DNA microarray data by real-time PCR. The up-regulation (HMOX1, TGFBI, LY6D, and S100P) and down-regulation (EIF4EBP2) of genes associated with annexin A11 expression (ANXA11) and the dynamic response of gene expression to cisplatin treatment (DHRS2 and PCSK9) were validated using real-time PCR. N represents control cells and R represents RNAi cells. For each individual gene, the expression levels at different time points were normalized to the control sample (N, PCR, 0 h). In addition, the relative mRNA expression levels were normalized to GAPDH expression. Each gene was amplified in triplicate, and each experiment was performed three times. *P<0.05, R versus N. **P<0.05, either [R2 (or R3 or R4) vs R1] or [N2 (or N3 or N4) vs N1]. (B) Suppression of annexin A11 upregulated HMOX1 and LY6D protein expressions. Immunoblot analysis was performed to confirm the suppression of annexin A11 protein expressions in the cells. The up-regulations of HMOX1 and LY6D protein expression levels in R1, R2, R3, and R4 compared with N1, N2, N3, and N4 were demonstrated. β-Actin was taken as an additional control for equal sampling in immunoblot analysis. (C) Annexin A11 immunointensity inversely correlated with HMOX1 immunoreactivity in ovarian cancer patients. Two representative pairs of tissue sections (left two sections from a primary tumor with low EDR and right two sections from a first recurrent tumor with extreme EDR) stained with two different antibodies are shown. Both sections of each pair were from similar areas of the same specimen. Original magnifications: upper panel, ×100; lower panel, ×400.

FIG. 5 is a Table (Table 1) that shows genes altered upon expression of Annexin A11.

### DETAILED DESCRIPTION OF THE INVENTION

As used herein, the term “cancer” is used to mean a condition in which a cell in a patient's body undergoes abnormal, uncontrolled proliferation. Thus, “cancer” is a cell-proliferative disorder. Non-limiting examples of cancers include breast cancer, cervical cancer, prostate cancer, colon cancer, lung cancer, skin cancer, melanoma or any other type of cancer.

The terms “array” or “matrix” refer to an arrangement of addressable locations or “addresses” on a device. The locations can be arranged in two-dimensional arrays, three-dimensional arrays, or other matrix formats. The number of locations may range from several to at least hundreds of thousands. Most importantly, each location represents a totally independent reaction site. A “nucleic acid array” refers to an array containing nucleic acid probes, such as oligonucleotides or larger portions of genes.

“Biological activity” or “bioactivity” or “activity” or “biological function,” which are used interchangeably, herein mean an effector or antigenic function that is directly or indirectly performed by a polypeptide (whether in its native or denatured conformation), or by any subsequence thereof. Biological activities include binding to polypeptides, binding to other proteins or molecules, activity as a DNA binding protein, as a transcription regulator, ability to bind damaged DNA, etc. A bioactivity can be modulated by directly affecting the subject polypeptide. Alternatively, a bioactivity can be altered by modulating the level of the polypeptide, such as by modulating expression of the corresponding gene.

The term “sample” or “biological sample,” as used herein, refers to a sample obtained from an organism or from components (e.g., cells) of an organism. The sample may be of any biological tissue or fluid. The sample may be a sample which is derived from a patient. Such samples include, but are not limited to, sputum, blood, blood cells (e.g., white cells), tissue or biopsy samples (e.g., tumor biopsy), urine, peritoneal fluid, and pleural fluid, or cells therefrom. Biological samples may also include sections of tissues such as frozen sections taken for histological purposes. The terms refer to a sample of tissue or fluid isolated from an individual, preferably suspected of being afflicted with, or at risk of developing cancer. Such samples are primary isolates (in contrast to cultured cells) and may be collected by a non-invasive means, including, but not limited to, fine needle aspiration, needle biopsy, or another suitable means recognized in the art. Alternatively, the “sample” may be collected by an invasive method, including, but not limited to, surgical biopsy.

The term “biomarker” or “marker” encompasses a broad range of intra- and extra-cellular events as well as whole-organism physiological changes. Biomarkers may be represent essentially any aspect of cell function, for example, but not limited to, levels or rate of production of signaling molecules, transcription factors, metabolites, gene transcripts as well as post-translational modifications of proteins. Biomarkers may include whole genome analysis of transcript levels or whole proteome analysis of protein levels and/or modifications.

A biomarker may also refer to a gene or gene product which is up- or down-regulated in a compound-treated, diseased cell of a subject having the disease compared to an untreated diseased cell. That is, the gene or gene product is sufficiently specific to the treated cell that it may be used, optionally with other genes or gene products, to identify, predict, or detect efficacy of a small molecule. Thus, a biomarker is a gene or gene product that is characteristic of efficacy of a compound in a diseased cell or the response of that diseased cell to treatment by the compound. In specific embodiments, the biomarkers of the invention are those polypeptides that are differentially expressed in cancerous samples when compared to non-cancerous samples. In a specific embodiment, the biomarker of the invention is annexin A11.

A nucleotide sequence is “complementary” to another nucleotide sequence if each of the bases of the two sequences match, that is, are capable of forming Watson-Crick base pairs. The term “complementary strand” is used herein interchangeably with the term “complement.” The complement of a nucleic acid strand may be the complement of a coding strand or the complement of a non-coding strand.

The term “cancer” includes, but is not limited to, solid tumors, such as cancers of the breast, respiratory tract, brain, reproductive organs, digestive tract, urinary tract, eye, liver, skin, head and neck, thyroid, parathyroid, and their distant metastases. The term also includes lymphomas, sarcomas, and leukemias.

“Hybridization” refers to any process by which a strand of nucleic acid binds with a complementary strand through base pairing. For example, two single-stranded nucleic acids “hybridize” when they form a double-stranded duplex. The region of double-strandedness may include the fill-length of one or both of the single-stranded nucleic acids, or all of one single-stranded nucleic acid and a subsequence of the other single-stranded nucleic acid, or the region of double-strandedness may include a subsequence of each nucleic acid. Hybridization also includes the formation of duplexes which contain certain mismatches, provided that the two strands are still forming a double-stranded helix. “Stringent hybridization conditions” refers to hybridization conditions resulting in essentially specific hybridization.

The term “isolated,” as used herein, with respect to nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, culture medium when produced by recombinant DNA techniques, or chemical precursors or other chemicals when chemically synthesized. Moreover, an “isolated nucleic acid” may include nucleic acid fragments which are not naturally occurring as fragments and would not be found in the natural state. The term “isolated” is also used herein to refer to polypeptides which are substantially free of other cellular proteins and is meant to encompass both purified and recombinant polypeptides.

As used herein, the term “level of expression” refers to the measurable expression level of a given polypeptide or nucleic acid molecule. The level of expression of the polypeptide or nucleic acid is determined by methods well known in the art. The term “differentially expressed” or “differential expression” refers to an increase or decrease in the measurable expression level of a given polypeptide or nucleic acid. Absolute quantification of the level of expression of a polypeptide or nucleic acid may be accomplished by comparing the level to that of a control. The control can be an average amount of the molecule in a statistically significant number of samples, or can be compared to a the level of the molecule in a non-cancerous sample.

As used herein, the term “nucleic acid” refers to polynucleotides such as deoxyribonucleic acid (DNA) and, where appropriate, ribonucleic acid (RNA). The term should also be understood to include, as equivalents, analogs of either RNA or DNA made from nucleotide analogs and, as applicable to the embodiment being described, single-stranded (sense or antisense) and double-stranded polynucleotides. Chromosomes, cDNAs, mRNAs, rRNAs, and ESTs are representative examples of molecules that may be referred to as nucleic acids.

The term “oligonucleotide” as used herein refers to a nucleic acid molecule comprising, for example, from about 10 to about 1000 nucleotides. Oligonucleotides for use in the present invention are preferably from about 15 to about 150 nucleotides, more preferably from about 150 to about 1000 in length. The oligonucleotide may be a naturally occurring oligonucleotide or a synthetic oligonucleotide. Oligonucleotides may be prepared by the phosphoramidite method (Beaucage and Carruthers, Tetrahedron Lett. 22:1859-62, 1981), or by the triester method (Matteucci, et al., J. Am. Chem. Soc. 103:3185, 1981), or by other chemical methods known in the art.

The term “protein” is used interchangeably herein with the terms “peptide” and “polypeptide.”

As used herein, the term “cell-proliferative disorder” denotes malignant as well as non-malignant (or benign) disorders. This term further encompasses hyperplastic disorders. The cells comprising these proliferative disorders often appear morphologically and genotypically to differ from the surrounding normal tissue. As noted above, cell-proliferative disorders may be associated, for example, with chemoresistance. Expression of a biomarker of the invention, e.g., annexin A11 may be indicative of chemoresistance. The biomarkers of the invention, e.g., annexin A11, also provide information to the clinician as to the likelihood of recurrence of cancer. The finding that a subject has altered levels of a biomarker of the invention can influence the course of treatment that subject receives.

As used herein, the term “chemotherapeutic agents” refers to chemicals useful for the treatment of cell proliferative disorders. Chemotherapeutic agents may be categorized by their mechanism of action into, for example, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes—dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

In preferred embodiments of the invention, the chemotherapeutic agent to which the subject becomes resistant to is a platinum based therapeutic, e.g., Carboplatin, Cisplatin, Oxaliplatin, BBR3464, Satraplatin. In a specific embodiment, the chemotherapeutic agent is cisplatin.

As used herein, the term, “chemoresistant” refers to subjects who fail to respond to the action of one or more chemotherapeutic agents. Most subjects are not chemoresistant at the beginning of treatment but may become so after a period of treatment. In specific embodiments, subjects that are chemoresistant are chemoresistant to platinum based therapeutics. In a particular embodiment, the subjects are chemoresistant to cisplatin.

### Methods of Detecting the Biomarkers

The instant invention is based on the finding that certain molecules are differentially expressed in cells that have become, or are becoming, chemoresistant. In order to determine if a cell is chemoresistant, of at risk of becoming chemoresistant, the instant invention provides methods for determining the level of the identified biomarkers in a biological sample. Specifically, the invention provides methods and compositions for determining the amount of a protein or nucleic acid biomarker of the invention in a biological sample. The biomarkers of the invention can be nucleic acid or polypeptide biomarkers. In a preferred embodiment, the biomarkers are polypeptides.

In certain preferred embodiments, the biomarkers are selected from PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, PDZD2, H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11.

In clinical applications, human tissue samples may be screened for the presence and/or absence of biomarkers identified herein. Such samples could consist of needle biopsy cores, surgical resection samples, lymph node tissue, or serum. For example, these methods include obtaining a biopsy, which is optionally fractionated by cryostat sectioning to enrich tumor cells to about 80% of the total cell population. In certain embodiments, nucleic acids extracted from these samples may be amplified using techniques well known in the art. The levels of selected markers detected could be compared with statistically valid normal tissue samples.

In one embodiment, the diagnostic method comprises determining whether a subject has an abnormal nucleic acid and/or protein level of the biomarkers, such as by Northern blot analysis, reverse transcription-polymerase chain reaction (RT-PCR), in situ hybridization, immunoprecipitation, Western blot hybridization, or immunohistochemistry. According to the method, cells may be obtained from a subject and the levels of the biomarkers, protein, or nucleic acid level, are determined and compared to the level of these markers in a healthy subject. An abnormal level of the biomarker polypeptide or nucleic acid levels is indicative of chemoresistance.

Accordingly, in one aspect, the invention provides probes and primers that are specific to the unique nucleic acid markers disclosed herein. Accordingly, the nucleic acid probes comprise a nucleotide sequence at least 10 nucleotides in length, preferably at least 15 nucleotides, more preferably, 25 nucleotides, and most preferably at least 40 nucleotides, and up to all or nearly all of the coding sequence which is complementary to a portion of the coding sequence of a marker nucleic acid sequence.

The invention further provides a method of determining whether a sample obtained from a subject possesses an abnormal amount of a biomarker of the invention comprising (a) obtaining a sample from the subject, (b) quantitatively determining the amount of the biomarker in the sample, and (c) comparing the amount of the marker polypeptide so determined with a known standard or to a control, thereby determining whether the sample obtained from the subject possesses an abnormal amount of the marker polypeptide. Such marker polypeptides may be detected by immunohistochemical assays, dot-blot assays, ELISA, and the like.

Immunoassays are commonly used to quantitate the levels of proteins in cell samples, and many other immunoassay techniques are known in the art. The invention is not limited to a particular assay procedure, and therefore, is intended to include both homogeneous and heterogeneous procedures. Exemplary immunoassays which may be conducted according to the invention include fluorescence polarization immunoassay (FPIA), fluorescence immunoassay (FIA), enzyme immunoassay (EIA), nephelometric inhibition immunoassay (NIA), enzyme-linked immunosorbent assay (ELISA), and radioimmunoassay (RIA). An indicator moiety, or label group, may be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

In another embodiment, the level of the encoded product, or alternatively the level of the polypeptide, in a biological fluid (e.g., blood or urine) of a patient may be determined as a way of monitoring the level of expression of the marker nucleic acid sequence in cells of that patient. Such a method would include the steps of obtaining a sample of a biological fluid from the patient, contacting the sample (or proteins from the sample) with an antibody specific for an encoded marker polypeptide, and determining the amount of immune complex formation by the antibody, with the amount of immune complex formation being indicative of the level of the marker encoded product in the sample. This determination is particularly instructive when compared to the amount of immune complex formation by the same antibody in a control sample taken from a normal individual or in one or more samples previously or subsequently obtained from the same person.

The term “antibody” as used herein includes antibodies that react with a biomarker of the invention or with one or more peptide fragments of a biomarker of the invention. The term “antibodies” is also intended to include parts thereof such as Fab, Fv fragments as well as antibodies that react with the overlapping regions of one or more of the peptide fragments of the invention and recombinantly produced fragments and fusion products of antibody fragments (including multivalent and/or multi-specific). The term “antibodies” is also intended to include antibodies to receptors specific for one or more of the peptide fragments of the invention. Antibodies can be fragmented using conventional techniques and the fragments screened for utility in the same manner as described above. Antibodies may be used either for screening for diagnostic purposes or in order to identify additional peptide fragments, mimetics, variants and inhibitors of the invention.

The term “autoantibody” refers to an antibody obtained from an individual or animal and which is reactive to a normal cellular antigen(s) or a self-antigen from the same individual or animal.

Conventional methods can be used to prepare the antibodies. For example, by using a peptide of the invention, polyclonal antisera or monoclonal antibodies can be made using standard methods. This invention also contemplates chimeric antibody molecules, made by methods known to those skilled in the art.

The antibodies may be labeled with a detectable marker including various enzymes, fluorescent materials, luminescent materials and radioactive materials as is known to those skilled in the art.

Antibodies reactive against naturally occurring biomarkers of the invention and fragments thereof (e.g., enzyme conjugates or labeled derivatives) may be used to detect a biomarker of the invention, including the peptide sequence in various samples, such as tissue or body fluid samples. For example, they may be used in any known immunoassays and immunological methods that rely on the binding interaction between an antigenic determinant of a protein of the invention and the antibodies. Examples of such assays are radioimmunoassays, Western immunoblotting, enzyme immunoassays (e.g. ELISA), immunofluorescence, immunoprecipitation, latex agglutination, and immunohistochemical tests. Thus, the antibodies may be used to identify or quantify the amount of a biomarker of the invention in a sample and thus may be used as a diagnostic indicator of chemoresistance.

A sample may be tested for the presence or absence of a biomarker of the invention by contacting the sample with an antibody specific for an epitope, e.g., an epitope of any one of PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, PDZD2, H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11.

Preferably, the antibody is capable of being detected after it becomes bound to a biomarker of the invention in the sample, and assaying for antibody bound to a biomarker of the invention in the sample.

In the method of the immunoassay, a predetermined amount of a biological sample or concentrated sample is preferably mixed with antibody or labelled antibody. The amount of antibody used in the method is dependent upon the labelling agent chosen. The amount of a biomarker of the invention bound to antibody or labelled antibody may then be detected by methods known to those skilled in the art. The sample or antibody may be insolubilized, for example, the sample or antibody can be reacted using known methods with a suitable carrier. Examples of suitable carriers are Sepharose or agarose beads. When an insolubilized sample or antibody is used, a biomarker of the invention bound to antibody or unreacted antibody is isolated by washing. For example, when the sample is blotted onto a nitrocellulose membrane, the antibody bound to a biomarker of the invention is separated from the unreacted antibody by washing with a buffer, for example, phosphate buffered saline (PBS) with bovine serum albumin (BSA).

When labeled antibody is used, the presence of a biomarker of the invention can be determined by measuring the amount of labeled antibody bound in the sample. The appropriate method of measuring the labeled material is dependent upon the labeling agent.

The methods of the invention may be performed on any related tissue or body fluid sample. In one embodiment, the sample is preferably a ovarian tissue sample. Alternatively, the methods of the invention can be performed on a body fluid sample selected from the group consisting of blood, plasma, serum, fecal matter, urine, semen, seminal fluid or plasma.

Polyclonal and monoclonal antibodies of the invention are immunoreactive with a biomarker of the invention or immunogenic fragments of a biomarker of the invention.

The term “antibody” also includes any synthetic or genetically engineered protein that is functionally capable of binding an epitopic determinant of a biomarker of the invention. It also refers to a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment.

An “antibody fragment” is a portion of an antibody such as F(ab')₂, F(ab)₂, Fab′, Fab, Fv, scFv (single chain Fv) and the like. Regardless of structure, an antibody fragment binds with to same antigen that is recognized by the intact antibody.

The term “antibody fragment” also includes any synthetic or genetically engineered protein that acts like an antibody by binding to a specific biomarker antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region. The Fv fragments may be constructed in different ways as to yield multivalent and/or multispecific binding forms. In the former case of multivalent, they react with more than one binding site against the specific epitope, whereas with multispecific forms, more than one epitope (either of the antigen or even against the specific antigen and a different antigen) is bound.

A “chimeric antibody” is a recombinant protein that contains the variable domains of both the heavy and light antibody chains, including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A “humanized antibody” is a recombinant protein in which the CDRs from an antibody from one species, e.g., a rodent antibody, is transferred from the heavy and light variable chains of the rodent antibody into human heavy and light variable domains. The constant domains of the antibody molecule are derived from those of a human antibody.

A “human antibody” is an antibody obtained from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. See for example, McCafferty et al., Nature 348:552-553 (1990) for the production of human antibodies and fragments thereof in vitro, from immunoglobulin variable domain gene repertoires from unimmunized donors. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see e.g., Johnson and Chiswell, Current Opinion in Structural Biol. 3:5564-571 (1993).

For purposes of the invention, an antibody or nucleic acid probe specific for an EPCA may be used to detect the presence of the a biomarker of the invention (in the case of an antibody probe) or polynucleotide (in the case of the nucleic acid probe) in biological fluids or tissues. Oligonucleotide primers based on any coding sequence region of a biomarker of the invention are useful for amplifying DNA or RNA, for example by PCR. The term “amplification” as used herein, relates to the production of additional copies of a nucleic acid sequence. Amplification is generally carried out using polymerase chain reaction (PCR) technologies that are well known in the art. (See, e.g., Dieffenbach, C. W. and G. S. Dveksler (1995), PCR Primer, a Laboratory Manual, Cold Spring Harbor Press, Plainview, N.Y., pp. 1-5). Any specimen containing a detectable amount of EPCA antigen can be used. A preferred sample of this invention is tissue taken from the prostate. Alternatively, biological fluids which may contain cells of the prostate may be used.

Methods that directly compare the qualitative and quantitative protein content of tumor and normal cells are known in the art. These methods include immunoassays, one-dimensional and two-dimensional gel electrophoresis characterization, western blotting, matrix assisted laser desorption/time of flight (MALDI/TOF) mass spectrometry, liquid chromatography quadruple ion trap electrospray (LCQ-MS) and surface enhanced laser desorption ionization/time of flight (SELDI/TOF) mass spectrometry. These methods coupled with the laser capture microdissection method of Liotta et al. (WO 00/49410) can determine the protein characteristics of a biological sample. These methods can be used to determine the level of a biomarker of the invention in a sample, i.e., the level in a biological sample v. a control sample.

The present invention contemplates using the above-mentioned methods to compare the protein of the present invention in normal and test samples. Biomarkers of the invention can be used either alone or in combination with a ligand, such as a monoclonal antibody. For example, SELDI can be used in combination with a time-of-flight mass spectrometer (TOF) to provide a means to rapidly analyze a biomarker of the invention or peptide fragments thereof retained on a chip (Hutchens and Yip, Rapid Commun. Mass Spectrom. 7:576-580, 1993). SELDI/TOF can be applied to ligand-protein interaction analysis by covalently binding the target protein on the chip and using mass spectroscopy to analyze the small molecules that bind to the target protein (Worrall et al. Anal Biochem. 70:750-756, 1998).

The immunological processes of a human subject may produce auto-antibodies directed to the protein of the present invention, as a result of a cell proliferative disorder, e.g., cancer. These antibodies, directed to a self-derived protein, would be an autoantibodies by definition. As such, autoantibodies can be measured in body fluids or tissues by immunological in vitro diagnostic methods wherein the biomarker of the invention protein or antigenic fragments thereof can be used as target substrates. The detection of auto-antibodies may correlate with the pathological state of cancer and, therefore, would be useful for diagnostic purposes.

Auto-antibodies reactive with for example, and one of PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, PDZD2, H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11 can be measured by a variety of immunoassay methods. For a review of immunological and immunoassay procedures in general, see Basic and Clinical Immunology, 7th Edition, D. Stites and A. Terr (ed.), 1991; “Practice and Theory of Enzyme Immunoassays,” P. Tijssen, Laboratory Techniques in Biochemistry and Molecular Biology, Elsevier Science Publishers, B. V., Amsterdam (1985); and Harlow and Lane, Antibodies, A Laboratory Manual. The entire contents of these references are incorporated herein by reference.

The invention also provides methods of determining expression levels of various genes in the biological samples as described above and comparing the expression levels with the expression level in a control sample.

The method for determining the expression levels of genes is not particularly limited, and any of techniques for confirming alterations of the gene expressions mentioned above can be suitably used.

In an exemplary method, mRNA is prepared from a biological sample, and then reverse transcription is carried out with the resulting mRNA as a template. During this process, labeled cDNA can be obtained by using, for instance, any suitable labeled primers or labeled nucleotides.

### Methods of Treatment

In one embodiment, the invention provides methods and compositions for treating a cell-proliferative disorder, e.g., ovarian cancer. In one embodiment, the instant invention provides methods for treating a subject having ovarian cancer by administering to a subject an effective amount of a compound that inhibits the activity of autoantibodies to, for example, PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, PDZD2, H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11.

The instant invention provides detailed teachings that decreased levels of certain polypeptides result in the subject becoming chemoresistant, having a poor prognosis, a decreased length of survival, and/or a increased risk of recurrence. Accordingly, methods that increase the level of the polypeptide to near wild-type levels would be useful to treat these subjects.

In particular, the present invention features methods of determining if a subject has become or is at risk of becoming chemoresistant, comprising obtaining a biological sample from the subject, and measuring the level of one or more proteins selected from HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, and PDZD2, PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, and ARF3, wherein an increased level of one or more proteins is indicative that the subject is or will become chemoresistant.

In particular embodiments, certain proteins are increased at certain times following treatment with chemotherapy. For example, certain genes may be increased at least 8, 16, or 24 hours after a subject is treated with chemotherapy. In certain cases, protein expression may be increased at 8 hours, for example, and then decreased at 24 hours, for example. In other cases, protein expression may be increased at 16 hours, and then decreased at 24 hours. In other cases, protein expression may be increased at 8 hours and/or 16 hours, and still increased at 24 hours.

In certain embodiments, PLEKHM1, KRTAP3-1, MB2, DERP12, and ZA31P are increased at least 8 hours after a subject is treated with chemotherapy.

In other embodiments , PLEKHM1, A_{—}24_P932355, PCSK9, MB2, and ZA31P are increased at least 16 hours after a subject is treated with chemotherapy.

In other embodiments, PLEKHM1, A_{—}24_P932355, MB2, ZA31P, DERP12, RC1, JAK3, BC038245, HSPA2, SOST, METTL7A, NGEF, GPR30, GLRX, A_{—}23_P72014, L3MBTL, KCNMB4, GNAZ, PCSK9, AK096109, COL9A3, and ARF3 are increased at least 24 hours after a subject is treated with chemotherapy.

Annexin A11 gene expression may also be decreased.

The invention also features methods of determining if a subject has become or is at risk of becoming chemoresistant, comprising obtaining a biological sample from the subject, and measuring the level of one or more proteins selected from HIST1H2BM, LOC391566, EIF4EBP2, HISTH2BK, SCML1, and ANXA11, H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, and NAV3, wherein a decreased level of one or more proteins is indicative that the subject is or will become chemoresistant.

In particular embodiments, certain proteins are decreased at certain times following treatment with chemotherapy. For example, certain genes may be decreased at least 8, 16, or 24 hours after a subject is treated with chemotherapy. In certain cases, protein expression may be decreased at 8 hours, for example, and then increased at 16 or 24 hours, for example. In other cases, protein expression may be decreased at 16 hours, and then decreased at 24 hours.

In certain embodiments, H1F0 and PLEKHM1 are decreased at least 8 hours after a subject is treated with chemotherapy.

In other embodiments, SERPINB2, MX1, KRT6C, ISGF3G, IFI44, IFIT1, IFI44L, ADAMTS1, pTR7, DHRS2, IL-8, CXCL2, MIRH1, PLEKHM1, A_{—}24_P932355 and IL1R2 are decreased at least 16 hours after a subject is treated with chemotherapy.

In still other embodiments, PLEKHM1, H1F0, PLEKHM1, SERPINB2, MX1, KRT6C, ISGF3G, IFI44, IFIT1, IFI44L, ADAMTS1, pTR7, IL-8, CXCL2, MIRH1, IL1R2, ZCCHC2, UBE2E1, ZNF358, H1F0, KRT6C, KRT6A, ISGF3G, IFI44, MX1, IFIT1, IFI44L, MX2, FLJ20035, ATP8A2, pTR7, TNC, DHRS2, SNHG7, ILIR2, IL8, CXCL2, A_{—}24_P707102, SERPINB2, NAV3, A_{—}24_P932355 and ADAMTS1 are decreased at least 24 hours after a subject is treated with chemotherapy.

Annexin A11 gene expression may also be decreased.

In certain examples, PLEKHM1 and A_{—}24_P932355 may also be decreased when annexin A11 gene expression is also decreased.

In certain particular embodiments, the protein is HMOX1.

In a particular embodiment, subjects who are chemoresistant to one or more chemotherapeutics are administered a polynucleotide that results in increased expression of H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, and NAV3

In another particular embodiment, subjects who are chemoresistant to one or more chemotherapeutics are administered a polynucleotide that results in increased expression of annexin A11.

In a particular embodiment, the subject is chemoresistant to a platinum based chemotherapeutic. Platinum-based compounds, such as cisplatin and oxaliplatin, are the cornerstone in the treatment of testicular, ovarian, colorectal, lung, lymphoma and other cancers. Platinum-based compounds include, but are not limited to, Carboplatin, Cisplatin, Oxaliplatin, BBR3464, and Satraplatin. Platinum-based compounds may also include bis-platinates.

In a particular embodiment, the subject is chemoresistant to cisplatin.

The present invention also provides methods of determining if subject is likely to have a recurrence of cancer comprising measuring the level of one or more proteins as described herein, wherein a decreased level or an increased level of one or more proteins is indicative that the subject will have a recurrence of cancer.

The present invention also provides methods of prognosis, where a decreased level or an increased level of one or more proteins is indicative of disease progression in the subject.

The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5,219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell. Biol. (1994) 14:2411, and in Woffendin, Proc. Natl. Acad. Sci. (1994) 91:1581.

Further non-viral delivery suitable for use includes mechanical delivery systems such as the approach described in Woffendin et al., Proc. Natl. Acad. Sci. USA (1994) 91(24): 11581. Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

### EXAMPLES

It should be appreciated that the invention should not be construed to be limited to the examples that are now described; rather, the invention should be construed to include any and all applications provided herein and all equivalent variations within the skill of the ordinary artisan.

Annexin A11 is a member of the annexin superfamily of structurally related Ca2+-dependent phospholipid-binding proteins. Despite their structural similarities, annexins have diverse functions including cell division, apoptosis, Ca2+ signaling, growth regulation, and secretory function [9-11]. Annexin A11 contains a conserved structural element, four tandem annexin repeats, in which the Ca2+-binding sites are located; a unique N-terminal domain rich in glycine, proline, and tyrosine residues involved in binding to calcyclin (S100A6) and the apoptosis-linked protein ALG2 [12,13]. Previous studies have suggested that annexin A11 may play a role in cellular DNA synthesis and in cell proliferation as well as in membrane trafficking events such as exocytosis [14-18]. Several members of the annexin superfamily had been demonstrated to be involved in drug resistance in a variety of human cancers [19-22]. Different drugs may have different effects on the expression of certain proteins. Recently, the present inventors have shown that annexin A11 was downregulated in cisplatin-resistant ovarian cancer cells compared with their parental cells; expressions of annexin A11 were significantly lower in recurrent tumors than those in the primary ovarian cancers; a lower expression of annexin A11 was significantly associated with earlier recurrence of ovarian cancers; and annexin A11 immunoreactivity inversely correlated with in vitro cisplatin resistance in ovarian cancers [23].

To further elucidate the molecular mechanism underlying the observed association between annexin A11 and cisplatin resistance in ovarian cancer, the present functional study was carried out using small interfering RNA (siRNA) followed by various in vitro assays. To identify potential downstream annexin A11 associated targets, a comprehensive time course study of cisplatin response in ovarian cancer cells with/without suppression of annexin A11 expression using whole-genome oligonucleotide microarrays was performed in the examples described herein.

### Example 1

### Effect and Duration of Silencing of Annexin A11 Using siRNA

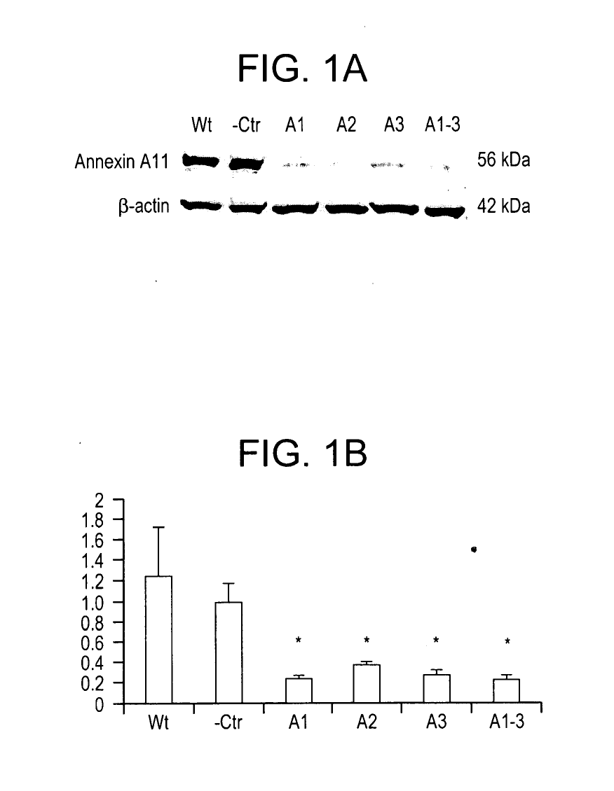

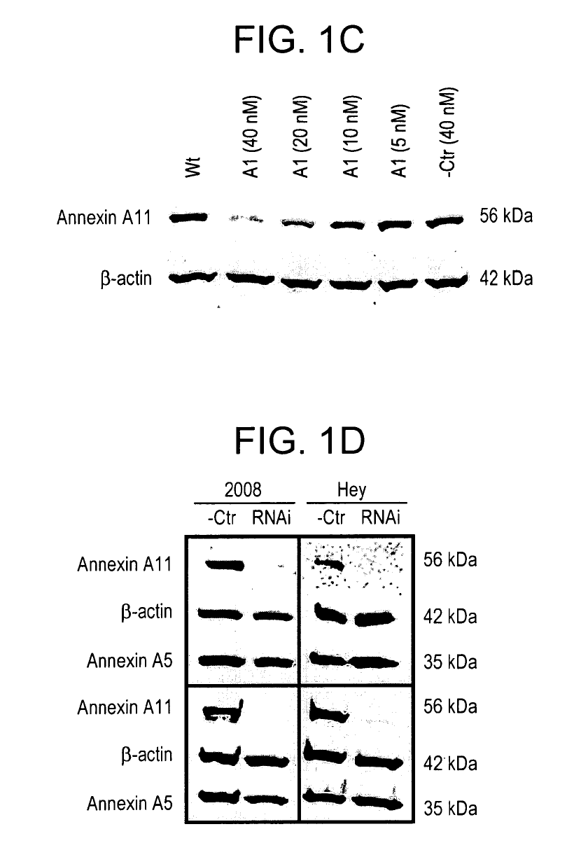

As shown in FIG. 1, A and B, after 3 days of siRNA transfection at the concentration of 40 nM, annexin A11-specific siRNA, either applied individually (A1, A2, and A3) or in combination (A1-3), significantly decreased annexin A11 mRNA and protein expression levels in 2008 cells. Quantitative real-time PCR revealed that there were about three-fold to four-fold of down-regulation in annexin A11 mRNA expression levels in RNAi-treated cells (A1, A2, A3, and A1-3) compared with negative control cells (-Ctr, P<0.05). Immunoblot analysis showed that there were only barely detectable annexin A11 protein expressions in RNAi-treated cells (A1, A2, A3, and A1-3) compared with annexin A11 strong expressions in negative control cells (-Ctr) and parental cells without treatment (Wt). Immunoblot analysis revealed a dose-dependent silencing effect of annexin A11 expression in RNAi (A1)-treated 2008 cells at the concentrations ranging from 5 to 40 nM (FIG. 1C). In addition, the experimental data demonstrated that the effect of silencing of annexin A11 protein expressions in 2008 and HEY cells lasted at least for 10 or 7 days after 3 or 2 days of siRNA transfection at the concentration of 40 nM, respectively (FIG. 1D).

### Example 2

### Knockdown of Annexin A11 Reduced Cancer Cell Proliferation and Colony Formation Ability

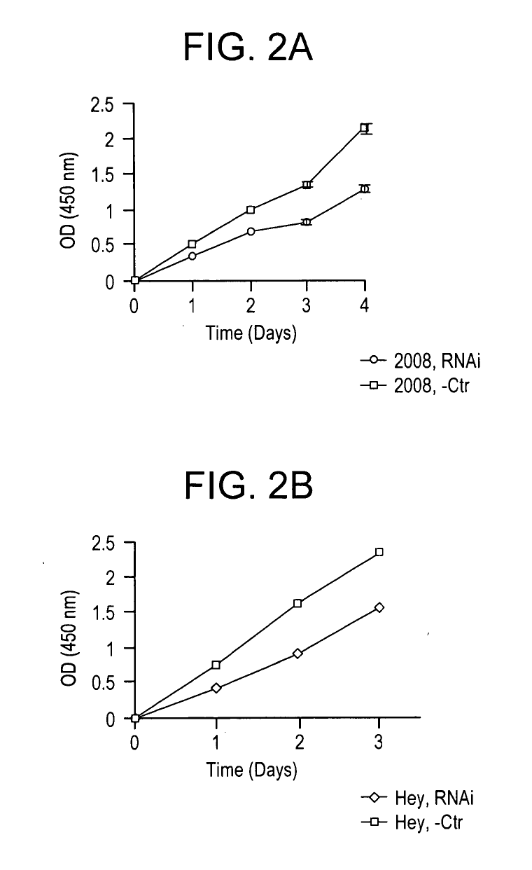

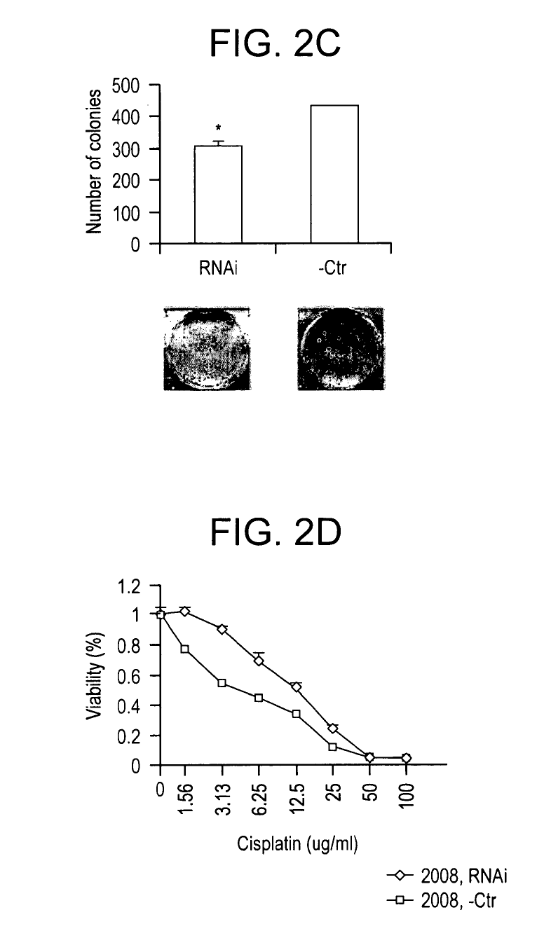

Cell growth and apoptosis are intimately related [24-28]. To determine the effect of annexin A11 on cell growth of ovarian cancer, cell proliferation assays and cell colony formation assays were performed after RNAi silencing of annexin A11 expression in 2008 and HEY cells. A significantly (P<0.05) slower rate of proliferation was observed (40% or 34% decreased) of the annexin A11-specific siRNA transfectants compared with that of the negative control transfectants in both 2008 and HEY cells (FIGS. 2, A and B). Suppression of annexin A11 expression also greatly damaged 2008 cell colony formation abilities (P<0.01; FIG. 2C). HEY cells did not form countable colonies during their growth process. These data suggested that annexin A11 plays an important role in cell proliferation of ovarian cancer.

### Example 3

### Epigenetic Silencing of Annexin A11 Conferred Chemoresistance to Ovarian Cancer Cells

Previously, the present inventors reported that annexin A11 is associated with cisplatin resistance and related to tumor recurrence in ovarian cancer patients [23]. To directly demonstrate the involvement of annexin A11 in cisplatin resistance of ovarian cancer cells, the cisplatin-sensitive 2008 cells were transfected with an annexin A11-specific siRNA or negative control followed by cell cytotoxicity assay. The sensitivities of the pair of cell lines to the cytotoxic effect of cisplatin were determined. Dose response curves were plotted on a semilog scale as the percentage of the control cell number, which was obtained from the sample without drug exposure. The experimental data showed that epigenetic silencing of annexin A11 expression significantly enhanced cisplatin resistance in 2008 cells (P<0.01; FIG. 2D). IC50 in two cell lines are 42 and 16 μM, respectively, with a 2.6-fold increase in RNAi cells compared with control cells. These data are consistent with the previous observation of an association between annexin A11 and cisplatin resistance in ovarian cancer [23].

### Example 4

### Dynamic Response of Gene Expression to Cisplatin Treatment and Annexin A11-Associated Gene Expression Alterations

To better understand the molecular mechanisms through which annexin A11 plays an important role in cell proliferation and drug resistance of ovarian cancer and to identify potential downstream annexin A11-associated targets, time course profiling of gene expressions was performed of both annexin A11-specific siRNA (R group) and negative control (N group) transfected ovarian cancer cells treated with cisplatin for different durations using the Agilent 44K whole genome oligo microarrays. Unsupervised analysis using principal component analysis indicated that cisplatin treatment has a major effect on global gene expression patterns of both cell lines at all time points (data not shown).

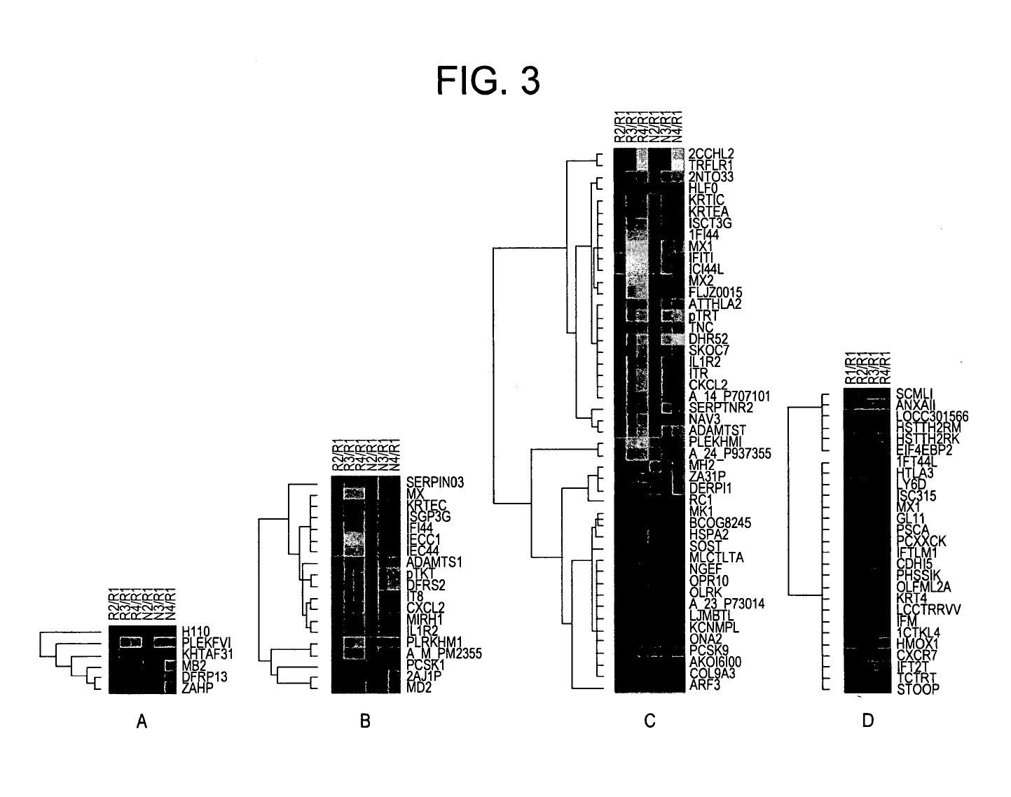

During the time course of cisplatin exposure, a total of 6 genes were either upregulated or downregulated at 8 hours after treatment with cisplatin, 19 after 16 hours and 47 after 24 hours in both groups of cells (FIG. 3, A-C). Most genes that altered at 8 hours (5/6) maintained their alterations of gene expression at 16 and/or 24 hours, representing a set of genes that were earlier and lasting responders to cisplatin exposure (FIG. 3A). H1F0, MB2, DERP12, and ZA31P showed consistent alterations (either up-regulation or down-regulation) of gene expression at all time points in both groups of cells. By 16 hours of cisplatin exposure, another major pattern of gene expression began to emerge. There were 15 genes that were altered at 16 hours but not at 8 hours and maintained their alterations at 24 hours. Among them, 12 genes including MX1, KRT6C, ISGF3G, IFI44, IFIT1, IFI44L, ADAMTS1, pTR7, DHRS2, IL8, CXCL2, and IL1R2 showed consistent down-regulations of gene expression at 16 and 24 hours in both groups of cells, which were organized into a major cluster (FIG. 3B). SERPINB2 was increased at 8 hours but decreased at 16 and 24 hours in both groups of cells, whereas PCSK9 was decreased at 8 hours but increased at 16 and 24 hours in both groups of cells. By 24 hours, more genes were included into this major cluster of downregulated genes, and a new major cluster of upregulated genes including PCSK9 was also formed as shown in FIG. 3C, suggesting the establishment of a large gene expression program in response to cisplatin exposure. Interestingly, among these genes, both PLEKHM1 and A_{—}24_P932355 showed total different responses to cisplatin treatment in both groups of cells. They were decreased in R group cells at 8, 16, and 24 hours, whereas they increased in N group cells at 8, 16, and 24 hours. The above identified genes altered during the time course of cisplatin exposure include some genes involved in apoptosis (PCSK9, SERPINB2, MX1), cell cycle/cell proliferation (H1F0, IL8, ADAMTS1, ATP8A2, DHRS2, KRT6C), signal transduction (IL8, ZCCHC2, ARF3, JAK3, KCNMB4, NGEF, PLEKHM1, TNC, CXCL2, GNAZ, GPR30, MX1, SOST), transcription regulation (L3MBTL, ZNF358, ISGF3G), cell adhesion (IL8,TNC), cellmotility/migration (IL8, S100P), metabolism (DHRS2, PCSK9, METTL7A, UBE2E1), immune response (IL8, CXCL2, IL1R2, IFIT1, ISGF3G, MX1, MX2), and nucleotide binding (ARF3, ATP8A2, GNAZ, H1F0, HSPA2, JAK3, MX1, MX2, NAV3, ZCCHC2, ZAF358).

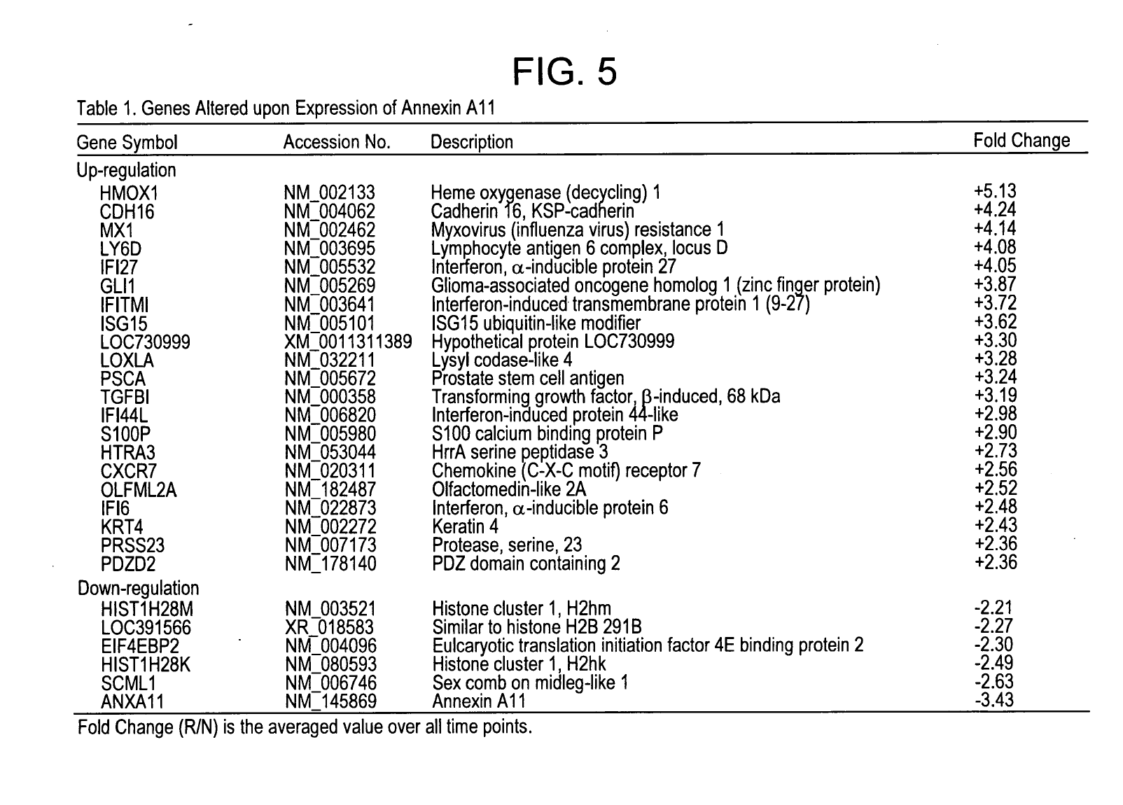

A total of 26 genes were identified as annexin A11-associated genes (FIG. 3D). Their expression levels were either increased (n=21) or decreased (n=5) after silencing of annexin A11. In this study, only genes with a fold up-regulation or down-regulation of at least 2 at every single time point were selected for validation. Table 1, shown in FIG. 5, lists these genes with averaged fold changes over all the time points, which were ordered accordingly. The identified annexin A11-associated genes include some genes involved in apoptosis/cell proliferation (HMOX1, MX1, GLI1, TGFBI, IFITM1, EIF4EBP2, HTRA3, IFI6, KRT4), DNA binding (HMOX1, MX1, HIST1H2BM, HIST1H2BK), signal transduction (HMOX1, GLI1, CXCR7, EIF4EBP2, IFITM1), transcription regulation (HMOX1, GLI1, SCML1), cell adhesion (TGFBI, CDH16, LY6D, PDZD2), and immune response (IFI27, IFI6, ISG15, MX1). Other genes were either only one gene in one category or without available annotation of gene ontology. The design of the time course gene expressions profiling study did not include replicates to allow for proper estimate of false discovery rate for results in FIG. 3, A-C. However, for the results in FIG. 3D, the identification of annexin A11-associated genes, using sample label permutation, the average number of genes with at least two-fold changes at every single time point was 5.8, indicating an estimated potential false discovery rate of 22%.

### Example 5

### Validation of DNA Microarray Data Using Real-time PCR

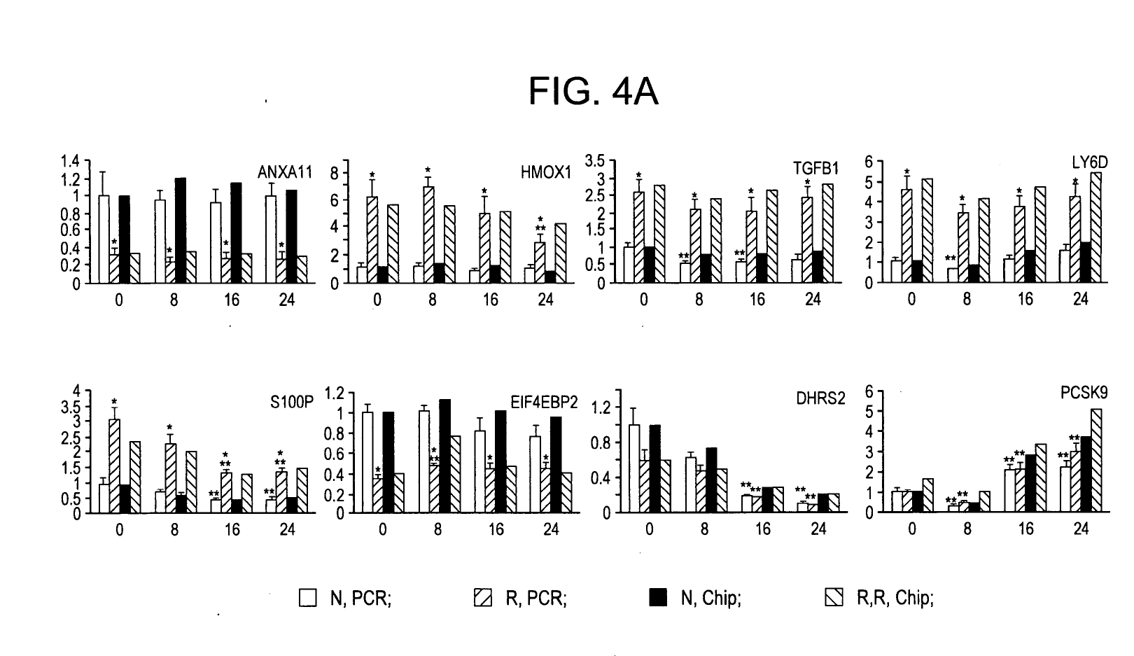

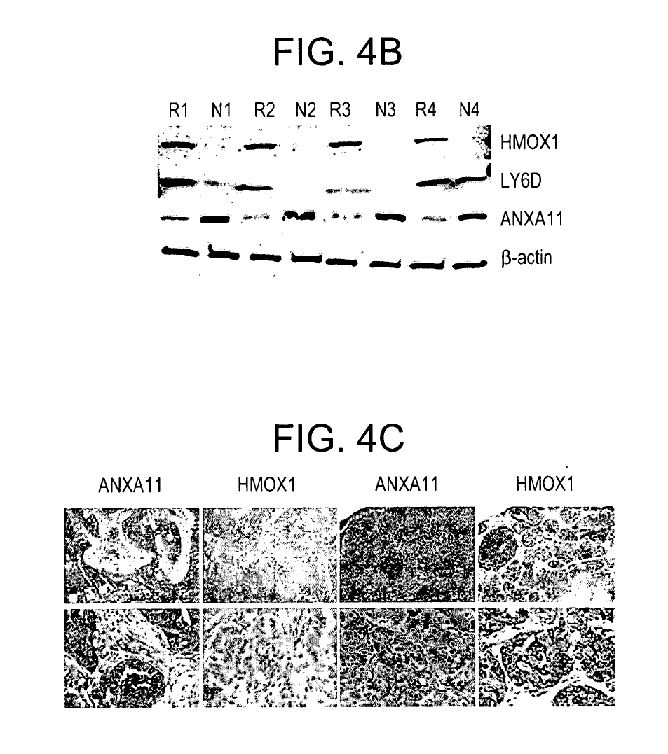

Before further effort to unravel the molecular pathways through which annexin A11 is involved in cell proliferation and drug resistance of ovarian cancer, the DNA microarray profiling data need to be validated using alternative platform. Real-time PCR assays were performed to independently determine mRNA expression levels on a set of genes that were representative of the above gene ontology classes: HMOX1, PCSK9, and EIF4EBP2 for apoptosis; HMOX1, TGFBI, DHRS2, and EIF4EBP2 for cell proliferation; TGFBI and LY6D for cell adhesion; HMOX1 for transcription regulation; HMOX1 and EIF4EBP2 for signal transduction; DHRS2 and PCSK9 for metabolism; HMOX1 for DNA binding; and S100P for cell migration. As shown in FIG. 4A and Table 3, below, the consistent up-regulation (HMOX1, TGFBI, LY6D, and S100P) and down-regulation (EIF4EBP2) of genes subjected to epigenetic silencing of annexin A11 (ANXA11) across all time points were well validated using real-time PCR.

TABLE 3

| Gene | | [symbol] | [symbol] | [symbol] | [symbol] | [symbol] | [symbol] | [symbol] | [symbol] | [symbol] | [symbol] |
|---|---|---|---|---|---|---|---|---|---|---|---|
| ANXA11 | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| HMOX1 | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| TGFBI | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| DHRS2 | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| PCSK9 | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] |

P values are shown for [symbol] [symbol] PCR [symbol] . [symbol] with P < .05 [symbol] [symbol] [symbol] [symbol] . P values with [symbol] response [symbol] gene [symbol] [symbol] [symbol] chip and PCR [symbol] . P values [symbol] [symbol] [symbol] [symbol] significance gene [symbol] [symbol] in PCR [symbol] . [symbol]
indicates data missing or illegible when filed

The down-regulation of DHRS2 that was one representative of the major cluster emerged at 16 and 24 hours after cisplatin exposure was verified in both groups of cells. PCSK9 that was identified as one of the major clusters of genes upregulated at the later time point(s) was also investigated using realtime PCR. The results showed dynamic responsive patterns of gene expression that were extremely similar to the microarray data, which was decreased at 8 hours but increased at 16 and 24 hours in both groups of cells. Overall, the real-time PCR results agreed well with the microarray data and confirmed that epigenetic silencing of annexin A11 in ovarian cancer cells followed by cisplatin exposure led to significant changes in the expression of genes involved in apoptosis, cell cycling/proliferation, cell adhesion, cell migration, transcription regulation, and signal transduction.

### Example 6

### Suppression of Annexin A11 Upregulated HMOX1 and LY6D Protein Expressions

According to the DNA microarray results, HMOX1 and LY6D were consistently increased by approximately 5.13- or 4.08-fold, respectively, in cells subjected to epigenetic silencing of annexin A11 across all time points. Using immunoblot analysis, the suppressions of annexin A11 protein expressions in the R group cells compared with those in the N group cells were confirmed. Immunoblot analysis showed that suppression of annexin A11 also upregulated HMOX1 and LY6D protein expression levels in R1, R2, R3, and R4 compared with N1, N2, N3, and N4, respectively (FIG. 4B).

### Example 7

### Annexin A11 Immunointensity Inversely Correlated with HMOX1 Immunoreactivity in Ovarian Cancer Patients

Owing to the extensive involvement of HMOX1 in different cellular processes including cell proliferation and apoptosis, the correlation of protein expressions between annexin A11 and HMOX1 in 150 ovarian carcinoma tissues was further evaluated with IHCstaining. HMOX1 immunoreactivity was observed in the cytoplasm of tumor cells (FIG. 4C) and significantly inversely correlated with annexin A11 immunointensity in 142 primary and first recurrent ovarian cancer patients (P=0.04; FIG. 4C and Table 2, shown below).

TABLE 2
Annexin A11 Immunointensity Correlates Inversely with
HMOX1 Immunoreactivity in Ovarian Cancer Patients

| | ANXA11 | | | | |
|---|---|---|---|---|---|
| HMOX1 | Negative | Weak | Moderate | [symbol] | Total |
| Negative | 5 (41.7%) | 19 (30.2%) | 23 (5 [symbol] %) | 13 (46.4%) | 60 |
| Postive | 7 (58.3%) | 44 ( [symbol] %) | 16 (41%) | 15 (53.6%) | 82 |
| Total | 12 | 63 | 39 | 28 | 142 (P = .04) |
| Negative | 4 (50%) | 5 (16.7%) | 6 (66.7%) | 3 (60%) | 18 |
| Positive | 4 (50%) | 25 (83.3%) | 3 (33.3%) | 2 (40%) | 34 |
| Total | 8 | 30 | 9 | 5 | 52 (P = .01) |

There is an inverse correlation of protein expression between annexin A11 and HMOX1 in 142 primary and first [symbol] ovarian cancer patients (P = .04). This inverse correlation [symbol] even more significantly in 52 first (P = .01). [symbol]
indicates data missing or illegible when filed

This inverse correlation exists even more significantly in 52 first recurrent tumors (P=0.01; Table 2). In addition, among 81 tumors for which the EDR results were available for analysis, HMOX1 immunoreactivity significantly positively correlated with in vitro cisplatin resistance (P=0.04; FIG. 4C). More specifically, there were approximately 60.6% of ovarian carcinomas with extreme and intermediate cisplatin resistance exhibited positive HMOX1 immunoreactivity, whereas only 37.5% of tumors with low cisplatin resistance showed positive HMOX1 immunoreactivity.

In the experiments and results described herein, it has been demonstrated that annexin A11 was directly involved in cell proliferation and cisplatin resistance of ovarian cancer. In particular, using RNAi techniques, it has been shown that knockdown of annexin A11 expression reduced cell proliferation and colony formation ability of ovarian cancer cells. Furthermore, it has been shown that epigenetic silencing of annexin A11 conferred cisplatin resistance to ovarian cancer cells. It has previously been shown that decreased expression of annexin A11 was characteristic for cisplatin-resistant ovarian cancer cells and may contribute to tumor recurrence in ovarian cancer patients [23]. The experimental results in this study are in agreement with the previous observation and further underscored the biological relevance of annexin A11 in the drug resistance of ovarian cancer.

Annexin A11 is a member of the annexin superfamily of Ca2+ and phospholipid-binding, membrane-associated proteins implicated in Ca2+ signal transduction processes associated with cell growth and differentiation [9-11]. Although diverse functions have been ascribed to annexins, there is no consensus about the role played by the annexin protein family as a whole [11]. The exact cellular functions of individual annexin members remain to be determined. Annexin A11 is ubiquitously expressed in a variety of tissues and cell types of eukaryotes, but its subcellular distribution varies considerably [14,17]. The nuclear localization of annexin A11 has been demonstrated to be cell type-specific and developmentally dependent [14]. Using recombinant human annexin A11-specific autoantibodies cloned by phage display, annexin A11 was found to be associated with the mitotic spindles and might play a role in cell division [17]. A combination of confocal and video time-lapse microscopy revealed that annexin A11 was required for midbody formation and completion of the terminal phase of cytokinesis [29]. A recent genome-wide association study identified ANXA11 as a new susceptibility locus for sarcoidosis and surmised that a depletion or dysfunction of annexin A11 may affect the apoptosis pathway in individuals with sarcoidosis and hence destroy the balance between apoptosis and survival of activated inflammatory cells [30]. In consistent with these observations, in this study, knockdown of annexin A11 expression resulted in a slower rate of cell growth in two ovarian cancer cell lines, 2008 and HEY, providing the first evidence that annexin A11 plays an important role in cell proliferation of ovarian cancer. In addition to the classic mechanisms, there are also several new molecular factors that have been linked to chemoresistance such as altered cell signaling pathways or presence of quiescent noncycling cells [22]. The cell cycle and apoptosis are intimately related, as evidenced by the central role of p53, both in cell cycle arrest and in the induction of apoptosis [25]. Conversely, this intimate relation was also demonstrated both in vitro and clinically; tumor cells that undergo a growth arrest or have a lower proliferation activity may be protected from apoptosis and may therefore be ultimately resistant to the cytotoxic agent [24,26]. The present results demonstrated that epigenetic silencing of annexin A11 expression reduced cell proliferation and conferred cisplatin resistance to ovarian cancer cells, suggesting the possibility that the observed association between annexin A11 and cisplatin resistance may be mediated through alterations in cell cycling/proliferation.

Although cancer cells with intrinsic or acquired cisplatin resistance have been analyzed to identify genomic or proteomic markers involved in drug resistance, the exact timing of transcriptional response to cisplatin treatment remains unclear. This longitudinal analysis of both annexin A11-specific siRNA and negative control-transfected ovarian cancer cells for their response to cisplatin treatment allowed the identification of some patterns of gene expressions in response to cisplatin exposure.

A set of genes altered at 8 hours and maintained their alterations of gene expression at 16 and 24 hours, representing earlier and lasting responders to cisplatin exposure. By 16 hours of cisplatin exposure, another major pattern of gene expression (down-regulation) began to emerge and maintained their alterations, with more genes included into this major cluster at 24 hours. Furthermore, the third major pattern of gene expression (up-regulation) was formed at 24 hours after initial downregulations of gene expressions at 8 hours of cisplatin exposure. These major patterns of gene expression suggested the establishment of a large gene expression program in response to cisplatin exposure. Many of these genes have been involved in apoptosis, cell cycle/proliferation, signal transduction, transcription regulation, cell adhesion, cell motility/migration, metabolism, and immune response. Tumor cells, in contrast to normal cells, respond to cisplatin exposure with transient gene expression to protect or repair their chromosomes. Some genes could serve as the master switch for turning on other genes in response to DNA damaging agents and play a major role in cisplatin resistance. PLEKHM1 was previously reported to be involved in colon cancer cells' response to cisplatin exposure [31]. Interestingly, in this study, PLEKHM1 showed totally different responses to cisplatin treatment in both groups of cells. In this study, a set of genes is also identified that are differentially expressed at all time points between two groups of cell lines, which represents the annexin A11-associated gene expression alterations. Many of these genes have been involved in apoptosis/cell proliferation, DNA binding, signal transduction, transcription regulation, and cell adhesion. Among them, the up-regulation of heme oxygenase 1 (HMOX1) or heat shock protein 32 (HSP32) seems particularly interesting because this inducible isoform of heme oxygenase has been shown to occur in various tumor tissues and contribute to tumor progression [32,33]. HMOX1 was reported to modulate different cellular functions including cytokine production, cell proliferation, and apoptosis and can exert unique cytoprotective effects [32-34]. It has previously been shown that HMOX1 attenuated the cisplatin-induced apoptosis of auditory cells [34] and that suppression of Nrf2-driven HMOX1 enhanced the chemosensitivity of lung cancer cells toward cisplatin [33]. In this study, HMOX1 immunoreactivity inversely correlated with annexin A11 immunointensity and positively correlated with in vitro cisplatin resistance in ovarian cancer patients, which suggested that HMOX1 may also collectively serve as a potential marker for ovarian cancer chemoresistance, and inhibition of intratumoral annexin A11-regulated HMOX1 activity may be a potential therapeutic strategy in human varian cancers. The extracellular matrix protein TGFBI induced microtubule stabilization and sensitized ovarian cancers to paclitaxel [35]. LY6D was reported to be a chemotherapy-induced antigen and has been used both as a therapeutic target and as a diagnostic marker for head and neck cancer [36-38]. S100P sensitizes ovarian cancer cells to carboplatin and paclitaxel in vitro [39]. IFITM1 was identified as a potent marker of cis-platinum response in esophageal cancer [40]. In this study, these annexin A11-associated genes were coordinately regulated to provide relatively different baselines in terms of gene expression and might be responsible for the observed phenotype changing of cancer cells.

The results and experiments presented herein demonstrate, in part, that annexin A11 is directly involved in cell proliferation and cisplatin resistance of ovarian cancer. Through a time course study of cisplatin response in ovarian cancer cells with/without suppression of annexin A11 expression, a set of differentially expressed genes was identified that is associated with annexin A11 expression and patterns of gene expressions in response to cisplatin exposure. Many of them such as HMOX1, TGFBI, LY6D, S100P, EIF4EBP2, DHRS2, and PCSK9 have been involved in apoptosis, cell cycling/proliferation, cell adhesion/migration, transcription regulation, and signal transduction. HMOX1 immunoreactivity inversely correlated with annexin A11 immunointensity and positively correlated with in vitro cisplatin resistance in ovarian cancer patients. Further characterization of these genes may contribute to a better understanding of the molecular mechanism through which annexin A11 plays an important role in cell proliferation and drug resistance of ovarian cancer. Manipulation of annexin A11 and its associated genes may represent a novel therapeutic strategy in human ovarian cancers.

### Methods

The above-described examples were carried out with, but not limited only to, the methods and materials described below.

### Cell Lines and Culture

Two cisplatin-sensitive ovarian cancer cell lines, 2008 and HEY, which were kindly provided by Dr. S. B. Howell, were used in this study [23]. All parental cell lines were maintained in drug-free RPMI-1640 medium (Invitrogen, Carlsbad, Calif.) supplemented with 10% (v/v) heat-inactivated fetal bovine serum (Hyclone, Logan, Utah) and 1% penicillin-streptomycin (Invitrogen) at 37° C. in a humidified atmosphere containing 5% CO2. All transfected cell lines were cultured in the same growth medium without antibiotics.

siRNA Knockdown of Annexin A11 Gene Expression

All stealth RNA interference (RNAi) sequences were purchased from Invitrogen. The three stealth RNAi that targeted different annexin A11 sequences were as follows: A1, GGCCGUGGUGAAAUGUCUCAAGAAU; A2, CCUCCUGGACAUCAGAUCAGAGUAU; and A3, GGGAUUACCGGAAGAUUCUGCUGAA. The stealth RNAi negative control duplex (medium GC) was used as a negative control. Transfection of annexin A11-specific siRNA and the negative control was performed using Lipofectamine 2000 (Invitrogen). The optimized dose and duration of RNAi silencing were experimentally determined. Briefly, cancer cells were seeded the day before siRNA transfection in either six-well plates or T25 flasks and were 30% to 50% confluent at the time of transfection. Stealth RNAi and Lipofectamine were diluted in Opti-MEM I Medium (Invitrogen), and 40 nM of the siRNA duplex was used in each transfection mixture. 2008 or HEY cells were transfected with one annexin A11-specific siRNA (A1 or A2 or A3) or a combination of three different siRNA at the equal amount (A1-3) or negative control for 2 to 3 days and were then harvested for the downstream experiments.

### Cell Proliferation Assay

Cell Counting Kit-8 (CCK-8; Dojindo, Gaithersburg, Md.) was used in cell proliferation assay. Briefly, 2008 and HEY cells were cultured in T25 flasks and transfected with annexin A11-specific siRNA (A1) or negative control for 3 days. Cells were then collected by trypsinization, counted by using a hemacytometer with trypan blue dye, and plated at 3000 viable cells per well into 96-well tissue culture plates in a final volume of 100 μl. Every 24 hours, a plate was subjected to assay by adding 10 μl of CCK-8 solution to each well, and the plate was further incubated for 4 hours at 37° C. The absorbance at 450 nm was measured with a microplate reader (EL 312e; Biotek Instruments, Winooski, Vt.). The experiment was performed in eight replicates.

### Cell Colony Formation Assay

2008 and HEY cells were cultured in T25 flasks and transfected with A1 or negative control for 3 days. Cells were then collected, counted, and plated at 3000 viable cells per well into six-well plates. Six days after plating, cells were fixed with methanol and stained with 0.1% crystal violet, and colonies were counted under the light microscope. The experiment was performed in six replicates.

### Cell Cytotoxicity Assay

2008 cells were cultured in T25 flasks and transfected with Al or negative control for 3 days. Cells were then collected, counted, and plated at 3000 viable cells per well into 96-well plates in a final volume of 100 μl. After incubating overnight, cells were treated with various concentrations of cisplatin diluted in 100 μl of conditioned medium (the final concentrations of cisplatin were 0, 1.56, 3.13, 6.25, 12.5, 25, 50, and 100 μg/ml). After incubating for 72 hours, the plates were assayed by CCK-8 as above. The experiment was performed in four replicates.

### Time Course Experiment of Annexin A11-Associated Cisplatin Response and Sample Preparations

2008 cells were cultured in T150 flasks and transfected with A1 or negative control for 2 days. Cells were then collected, counted, and placed into 100-mm dishes. After incubating overnight, transfected cells were at 50% confluence and treated with 10 μM cisplatin (Sigma-Aldrich, St. Louis, Mo.) for 0, 8, 16, and 24 hours and then harvested in two portions for both total RNA and total protein extractions at every single time point. The optimized dose and duration of cisplatin treatment were experimentally determined in a previous study [23]. Total RNA was isolated using TRIZOL reagent (Invitrogen) followed by RNeasy mini kit with DNase on-column digestion (Qiagen, Valencia, Calif.). RNA was quantified with NanoDrop ND-1000 followed by quality assessment with the 2100 Bioanalyzer (Agilent Technologies, Santa Clara, Calif.) according to manufacturer's protocol.

### Agilent Whole-Genome Oligo Microarray

Total RNA was labeled using Agilent Low RNA Input Fluorescent Linear Amplification Kit (Agilent) following the manufacturer's instruction with minor modifications. Briefly, 0.4 μg of RNA was reverse transcribed into cDNA by MMLV-RT using an oligo dT primer (System Biosciences, Mountain View, Calif.) that incorporated a T7 promoter sequence. The cDNA was then used as a template for in vitro transcription in the presence of T7 RNA polymerase and cyanine-3-labeled CTPs (Perkin Elmer Life Sciences, Boston, Mass.). RNA spike-in controls (Agilent) were added to RNA samples before amplification and labeling. The labeled cRNA was purified using the RNeasy mini kit (Qiagen). A total of 0.825 μg of each Cy3-labeled sample was used for hybridization on Agilent 4×44K whole human genome microarray at 65° C. for 17 hours in a hybridization oven with rotation. After hybridization, slides were washed and dried using stabilization and drying solution according to the Agilent microarray processing protocol. Slides were scanned using the Agilent Microarray Scanner controlled by Agilent Scan Control 7.0 software.

### Microarray Data Analysis

Microarray data were extracted with Agilent Feature Extraction 9.5.3.1 software and imported into GeneSpring GX 10 (Agilent). Normalization was done with all intensities higher than 5 by crossarray quantile normalization in log2 scale. Data were then transformed back to original scale for the remaining analysis. Features with intensities smaller than 300 at all time points were excluded from the analysis, and the resulting data were used for principal component analysis using MATLAB version 7.5 software. To identify genes that were differentially expressed at different time points, genes that were either upregulated or downregulated more than two-fold at 8, 16, or 24 hours compared with 0 hour in both cell lines after cisplatin exposure were selected. To identify genes that were differentially expressed between transfected cells with or without annexin A11 silencing, genes with a fold up-regulation or down-regulation of at least two at every single time point were chosen. The identified genes were then clustered and the heat maps representing gene expression at different time points were generated using the Cluster and TreeView software. Gene ontology analysis was performed using the Ingenuity pathway analysis program.

### Real-Time Reverse Transcription-Polymerase Chain Reaction

Total RNA was isolated from the different cancer cell lines using TRIZOL

(Invitrogen) according to the manufacturer's instructions. One microgram of total RNA was used to generate cDNA using the iScript cDNA Synthesis Kit (Bio-Rad, Hercules, Calif.). One microliter of the resulting cDNA was used in the subsequent polymerase chain reaction (PCR) in iQ SYBR Green Supermix (Bio-Rad), with the following cycles: 95° C. for 3 minutes followed by 50 cycles at 95° C. for 30 seconds, at 60° C. for 30 seconds, and at 72° C. for 55 seconds. An experiment consisted of triplicate amplification reactions for each gene product being analyzed. The GAPDH mRNA was used as an internal control for equal sampling of total RNA from one cell to another. The cycle threshold number (CT) was determined for each PCR using iQ5 Real-time PCR Detection System (Bio-Rad). The comparative CT method was used to calculate the relative abundance of a target transcript with regard to an internal control (GAPDH). Results are expressed as relative abundance of a specific mRNA between control and experimental sample (fold change, mean±SD). Sequences and product sizes for all of genes are shown in Table W1.

### Immunoblot Analysis

The denatured samples were electrophoresed on 4% to 15% gradient SDS-PAGE gels (Bio-Rad), electroblotted on nitrocellulose membranes (Bio-Rad), and probed with the respective antibodies against different targets. Both anti-annexin A11 (1:10,000) and anti-annexin A5 (1:2000) monoclonal antibodies were purchased from BD Biosciences (San Jose, Calif.). Rabbit anti-human HMOX1 polyclonal antibody (1:500) and mouse polyclonal anti-LY6D (1:500) were purchased from GenWay Biotech (San Diego, Calif.) and Novus Biologicals (Littleton, Colo.), respectively. The bound antibodies were visualized with horseradish peroxidase-conjugated secondary antibodies and enhanced chemiluminescence (Amersham, Pittsburgh, Pa.). Actin in the corresponding cell lysates was used as an additional control to show equal loading.

### Tissue Microarrays and Immunohistochemistry

In accordance with the human subject research guidelines of institutional review board, formalin-fixed, paraffin-embedded tissues were obtained from the Department of Pathology at Johns Hopkins Hospital. These included 150 ovarian carcinoma tissues, which are 90 primary tumors, 52 first recurrent tumors, and 8 second or third recurrent tumors. Detailed clinicopathologic characteristics of the study cohort have been previously described [23]. All patients underwent primary debulking surgery followed by platinum/paclitaxel-based combined chemotherapy. Tissue microarrays were constructed to facilitate immunohistochemistry (IHC) using EnVision+System-HRP kit (Dako, Carpinteria, Calif.) with an anti-annexin A11 monoclonal antibody (1:200; BD Biosciences) [23] and an anti-HMOX1 polyclonal antibody (1:200; BioVision, Mountain View, Calif.). The IHC staining of the protein were scored semiquantitatively as described previously [23]. In vitro cisplatin responses of tumors were assessed by the extreme drug resistance (EDR) assay (Oncotech, Tustin, Calif.) and have been previously described [23].

### Statistical Analysis

All of the statistical analyses were performed using the Statistica 6.1 (Statsoft, Tulsa, Okla.). Data were subjected to Student's unpaired t test or Fisher's exact test. Differences with P<0.05 were considered statistically significant.

TABLE 4
below shows primers used in the examples
and corresponding size.

| Primer | | Product Size |
|---|---|---|
| Name | Sequence | [symbol] |
| [symbol] | [symbol] | |
| [symbol] | [symbol] | 185 |
| [symbol] | [symbol] | |
| [symbol] | [symbol] | 153 |
| [symbol] | [symbol] | |
| [symbol] | [symbol] | 172 |
| [symbol] | [symbol] | |
| [symbol] | [symbol] | 154 |
| [symbol] | [symbol] | |
| [symbol] | [symbol] | 177 |
| [symbol] | [symbol] | |
| [symbol] | [symbol] | 153 |
| [symbol] | [symbol] | |
| [symbol] | [symbol] | 217 |
| [symbol] | [symbol] | |
| [symbol] | [symbol] | 188 |
| [symbol] | [symbol] | |
| [symbol] | [symbol] | 186 |

[symbol]

indicates data missing or illegible when filed

### REFERENCES

[1] Jemal A, Siegel R, Ward E, Hao Y, Xu J, Murray T, and Thun M J (2008). Cancer statistics, 2008. CA Cancer J Clin 58, 71-96.

[2] Holschneider C H and Berek J S (2000). Ovarian cancer: epidemiology, biology, and prognostic factors. Semin Surg Oncol 19,3-10.

[3] Bristow R E, Tomacruz R S, Armstrong D K, Trimble E L, and Montz F J (2002). Survival effect of maximal cytoreductive surgery for advanced ovarian carcinoma during the platinum era: a meta-analysis. J Clin Oncol 20, 1248-1259.

[4] Tummala M K and McGuire W P (2005). Recurrent ovarian cancer. Clin Adv Hematol Oncol 3, 723-736.

[5] Boulikas T and Vougiouka M (2003). Cisplatin and platinum drugs at the molecular level. (Review). Oncol Rep 10, 1663-1682.

[6] Siddik ZH (2003). Cisplatin: mode of cytotoxic action and molecular basis of resistance. Oncogene 22, 7265-7279.

[7] Fink D, Aebi S, and Howell S B (1998). The role of DNA mismatch repair in drug resistance. Clin Cancer Res 4, 1-6.

[8] Johnson S W, Laub P B, Beesley J S, Ozols R F, and Hamilton T C (1997). Increased platinum-DNA damage tolerance is associated with cisplatin resistance and crossresistance to various chemotherapeutic agents in unrelated human ovarian cancer cell lines. Cancer Res 57, 850-856.

[9] Brichory F M, Misek D E, Yim A M, Krause M C, Giordano T J, Beer D G, and Hanash S M (2001). An immune response manifested by the common occurrence of annexins I and II autoantibodies and high circulating levels of IL-6 in lung cancer. Proc Natl Acad Sci USA 98,9824-9829.

[10] Chetcuti A, Margan S H, Russell P, Mann S, Millar D S, Clark S J, Rogers J, Handelsman D J, and Dong Q (2001). Loss of annexin II heavy and light chains in prostate cancer and its precursors. Cancer Res 61, 6331-6334.

[11] Monastyrskaya K, Babiychuk E B, Hostettler A, Rescher U, and Draeger A (2007). Annexins as intracellular calcium sensors. Cell Calcium 41, 207-219.

[12] Fernandez M P, Jenkins N A, Gilbert D J, Copeland N G, and Morgan R O (1996). Sequence and chromosomal localization of mouse annexin XI. Genomics 37, 366-374.

[13] Furge L L, Chen K, and Cohen S (1999). Annexin VII and annexin XI are tyrosine phosphorylated in peroxovanadate-treated dogs and in platelet-derived growth factor-treated rat vascular smooth muscle cells. J Biol Chem 274, 33504-33509.

[14] Mamiya N, Iino S, Mizutani A, Kobayashi S, and Hidaka H (1994). Development related and cell-type specific nuclear localization of annexin XI: immunolocalization analysis in rat tissues. Biochem Biophys Res Commun 202, 403-409.

[15] Misaki Y, Pruijn G J, van der Kemp A W, and van Venrooij W J (1994). The 56K autoantigen is identical to human annexin XI. J Biol Chem 269, 4240-4246.

[16] Jorgensen CS, Levantino G, Houen G, Jacobsen S, Halberg P, Ullman S, Khamashta M A, Asmussen K, Oxholm P, Jorgensen M K, et al. (2000). Determination of autoantibodies to annexin XI in systemic autoimmune diseases. Lupus 9, 515-520.

[17] Farnaes L and Ditzel H J (2003). Dissecting the cellular functions of annexin XI using recombinant human annexin XI-specific autoantibodies cloned by phage display. J Biol Chem 278, 33120-33126.

[18] Williams L H, McClive P J, Van Den Bergen J A, and Sinclair A H (2005). Annexin XI co-localises with calcyclin in proliferating cells of the embryonic mouse testis. Dev Dyn 234, 432-437.

[19] Han E K, Tahir S K, Cherian S P, Collins N, and Ng S C (2000). Modulation of paclitaxel resistance by annexin IV in human cancer cell lines. Br J Cancer 83, 83-88.

[20] Wang Y, Serfass L, Roy M O, Wong J, Bonneau A M, and Georges E (2004). Annexin-I expression modulates drug resistance in tumor cells. Biochem Biophys Res Commun 314, 565-570.

[21] Le Moguen K, Lincet H, Deslandes E, Hubert-Roux M, Lange C, Poulain L, Gauduchon P, and Baudin B (2006). Comparative proteomic analysis of cisplatin sensitive IGROV1 ovarian carcinoma cell line and its resistant counterpart IGROV1-R10. Proteomics 6, 5183-5192.

[22] Stewart J J, White J T, Yan X, Collins S, Drescher C W, Urban N D, Hood L, and Lin B (2006). Proteins associated with cisplatin resistance in ovarian cancer cells identified by quantitative proteomic technology and integrated with mRNA expression levels. Mol Cell Proteomics 5, 433-443.

[23] Song J, Shih Ie M, Salani R, Chan D W, and Zhang Z (2007). Annexin XI is associated with cisplatin resistance and related to tumor recurrence in ovarian cancer patients. Clin Cancer Res 13, 6842-6849.

[24] Dimanche-Boitrel M T, Pelletier H, Genne P, Petit J M, Le Grimellec C, Canal P, Ardiet C, Bastian G, and Chauffert B (1992). Confluence-dependent resistance in human colon cancer cells: role of reduced drug accumulation and low intrinsic chemosensitivity of resting cells. Int J Cancer 50, 677-682.

[25] Kohn K W, Jackman J, and O'Connor P M (1994). Cell cycle control and cancer chemotherapy. J Cell Biochem 54, 440-452.

[26] Waldman T, Zhang Y, Dillehay L, Yu J, Kinzler K, Vogelstein B, and Williams J (1997). Cell-cycle arrest versus cell death in cancer therapy. Nat Med 3, 1034-1036.

[27] Itamochi H, Kigawa J, Akeshima R, Sato S, Kamazawa S, Takahashi M, Kanamori Y, Suzuki M, Ohwada M, and Terakawa N (2002). Mechanisms of cisplatin resistance in clear cell carcinoma of the ovary. Oncology 62, 349-353.

[28] Itamochi H, Kigawa J, Sugiyama T, Kikuchi Y, Suzuki M, and Terakawa N (2002). Low proliferation activity may be associated with chemoresistance in clear cell carcinoma of the ovary. Obstet Gynecol 100, 281-287.

[29] Tomas A, Futter C, and Moss S E (2004). Annexin 11 is required for midbody formation and completion of the terminal phase of cytokinesis. J Cell Biol 165, 813-822.

[30] Hofmann S, Franke A, Fischer A, Jacobs G, Nothnagel M, Gaede K I, Schurmann M, Muller-Quernheim J, Krawczak M, Rosenstiel P, et al. (2008). Genome-wide association study identifies ANXA11 as a new susceptibility locus for sarcoidosis. Nat Genet 40, 1103-1106.

[31] Duale N, Lindeman B, Komada M, Olsen A K, Andreassen A, Soderlund E J, and Brunborg G (2007). Molecular portrait of cisplatin induced response in human testis cancer cell lines based on gene expression profiles. Mol Cancer 6, 53.

[32] Marinissen M J, Tanos T, Bolos M, de Sagarra M R, Coso O A, and Cuadrado A (2006). Inhibition of heme oxygenase-1 interferes with the transforming activity of the Kaposi sarcoma herpesvirus-encoded G protein-coupled receptor. J Biol Chem 281, 11332-11346.

[33] Kim H R, Kim S, Kim E J, Park J H, Yang S H, Jeong E T, Park C, Youn M J, So H S, and Park R (2008). Suppression of Nrf2-driven heme oxygenase-1 enhances the chemosensitivity of lung cancer A549 cells toward cisplatin. Lung Cancer 60, 47-56.

[34] Kim H J, So H S, Lee J H, Lee J H, Park C, Park S Y, Kim Y H, Youn M J, Kim S J, Chung S Y, et al. (2006). Heme oxygenase-1 attenuates the cisplatin-induced apoptosis of auditory cells via down-regulation of reactive oxygen species generation. Free Radic Biol Med 40, 1810-1819.

[35] Ahmed A A, Mills A D, Ibrahim A E, Temple J, Blenkiron C, Vias M, Massie C E, Iyer N G, McGeoch A, Crawford R, et al. (2007). The extracellular matrix protein TGFBI induces microtubule stabilization and sensitizes ovarian cancers to paclitaxel. Cancer Cell 12, 514-527.

[36] de Bree R, Roos J C, Plaizier M A, Quak J J, van Kamp G J, den Hollander W, Snow G B, and van Dongen GA (1997). Selection of monoclonal antibody E48 IgG or U36 IgG for adjuvant radioimmunotherapy in head and neck cancer patients. Br J Cancer 75, 1049-1060.

[37] Nieuwenhuis E J, Leemans C R, Kummer J A, Denkers F, Snow G B, and Brakenhoff R H (2003). Assessment and clinical significance of micrometastases in lymph nodes of head and neck cancer patients detected by E48 (Ly-6D) quantitative reverse transcription-polymerase chain reaction. Lab Invest 83, 1233-1240.

[38] Rubinfeld B, Upadhyay A, Clark S L, Fong S E, Smith V, Koeppen H, Ross S, and Polakis P (2006). Identification and immunotherapeutic targeting of antigens induced by chemotherapy. Nat Biotechnol 24, 205-209.

[39] Wang Q, He Z, Gao J, Hu S, Huang M, Liu M, Zheng J, and Tang H (2008). S100P sensitizes ovarian cancer cells to carboplatin and paclitaxel in vitro. Cancer Lett 272, 277-284.

[40] Fumoto S, Shimokuni T, Tanimoto K, Hiyama K, Otani K, Ohtaki M, Hihara J, Yoshida K, Hiyama E, Noguchi T, et al. (2008). Selection of a novel drug-response predictor in esophageal cancer: a novel screening method using microarray and identification of IFITM1 as a potent marker gene of CDDP response. Int J Oncol 32, 413-423.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of determining if a subject has become or is at risk of becoming chemoresistant, comprising:
obtaining a biological sample from the subject; and
measuring the level of one or more proteins selected from the group consisting of: PLEKHM1, KRTAP3-1, MB2, DERP12, ZA31P, A_{—}242932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, and ARF3,
wherein an increased level of one or more proteins is indicative that the subject is or will become chemoresistant.

2. The method of claim 1, wherein PLEKHM1, KRTAP3-1, MB2, DERP12, and ZA31 P are increased at least 8 hours after a subject is treated with chemotherapy.

3. The method of claim 1, wherein PLEKHM1, A242932355, PCSK9, MB2, and ZA31P are increased at least 16 hours after a subject is treated with chemotherapy.

4. The method of claim 1, wherein PLEKHM1, A_{—}24_P932355, MB2, ZA31P, DERP12, RC1, JAK3, BC038245, HSPA2, SOST, METTL7A, NGEF, GPR30, GLRX, A_{—}23_P72014, L3MBTL, KCNMB4, GNAZ, PCSK9, AK096109, COL9A3, and ARF3 are increased at least 24 hours after a subject is treated with chemotherapy.

5. The method of claim 1, wherein annexin A11 gene expression is also decreased.

6. A method of determining if a subject has become or is at risk of becoming chemoresistant, comprising:
obtaining a biological sample from the subject; and
measuring the level of one or more proteins selected from the group consisting of H1F0, PLEKHM1, SERPINB2, MX1, KRT6C, ISGF3G, IFI44, IFIT1, IFI44L, ADAMTS1, pTR7, DHRS2, IL8, CXCL2, MIRH1, ILIR2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, and NAV3,
wherein a decreased level of one or more proteins is indicative that the subject is or will become chemoresistant.

7. The method of claim 6, wherein H1F0 and PLEKHM1 are decreased at least 8 hours after a subject is treated with chemotherapy.

8. The method of claim 6, wherein SERPINB2, MX1, KRT6C, ISGF3G, IFI44, IFIT1, IFI44L, ADAMTS1, pTR7, DHRS2, IL-8, CXCL2, MIRH1, PLEKHM1, A_{—}24_P932355 and IL1R2 are decreased at least 16 hours after a subject is treated with chemotherapy.

9-11. (canceled)

12. A method of determining if a subject has become or is at risk of becoming chemoresistant, comprising:
obtaining a biological sample from the subject; and
measuring the level of one or more proteins selected from the group consisting of: HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, and PDZD2,
wherein an increased level of the protein is indicative that the subject is or will become chemoresistant, or
A method of determining if a subject has become or is at risk of becoming chemoresistant, comprising:
obtaining a biological sample from the subject; and
measuring the level of one or more proteins selected from the group consisting of: HIST1H2BM, LOC391566, EIF4EBP2, HISTH2BK, SCML1, and ANXA11;
wherein a decreased level of the protein is indicative that the subject is or will become chemoresistant.

13-16. (canceled)

17. The method of claim 1, wherein the subject is chemoresistant to a platinum based chemotherapeutic.

18-19. (canceled)

20. The method of claim 1, wherein the subject has a cell proliferative disorder.

21-26. (canceled)

27. A method of determining if a subject having ovarian cancer has become, or is at risk of becoming chemoresistant, comprising:
obtaining a biological sample from the subject; and
measuring the level of one or more proteins selected from the group consisting of H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11 polypeptide in the sample,
wherein a decreased level of one or more proteins is indicative that the subject is or will become chemoresistant, or
A method of determining if a subject having ovarian cancer has become, or is at risk of becoming chemoresistant, comprising:
obtaining a biological sample from the subject; and
measuring the level of one or more proteins selected from the group consisting of: PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, and PDZD2 polypeptide in the sample,
wherein an increased level of one or more proteins is indicative that the subject is or will become chemoresistant.

28-35. (canceled)

36. A method of determining if subject is likely to have a recurrence of cancer comprising:
obtaining a biological sample from the subject; and
measuring the level of one or more proteins selected from the group consisting of H1 F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11 polypeptide in the sample,
wherein a decreased level of one or more proteins is indicative that the subject will have a recurrence of cancer, or
A method of determining if a subject is likely to have a recurrence of cancer comprising:
obtaining a biological sample from the subject; and
measuring the level of one or more proteins selected from the group consisting of: PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, and PDZD2 polypeptide in the sample;
wherein an increased level of one or more proteins is indicative that the subject will have a recurrence of cancer, or
A method of treating a subject having cancer comprising:
administering to the subject a nucleic acid molecule encoding one or more proteins selected from the group consisting of: PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, PDZD2, H1F0, PLEKHM1, SERPINB2, MX1, KRT6C, ISGF3G, IFI44, IFIT1, IFI44L, ADAMTS1, pTR7, DHRS2, IL8, CXCL2, MIRH1, ILIR2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11,
wherein the nucleic acid molecule is capable of producing the one or more polypeptides in the cells of the subject.

37-43. (canceled)

44. A method of determining the prognosis of a subject having cancer comprising:
obtaining a biological sample from the subject; and
measuring the level of one or more proteins selected from the group consisting of H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HISTIH2BM, LOC391566, EIF4EBP2, HISTH2BK, SCML1, and ANXA11 polypeptide in the sample;
wherein a decreased level of one or more proteins is indicative of a poor prognosis, or
A method of determining the prognosis of a subject having cancer comprising:
obtaining a biological sample from the subject; and
measuring the level of one or more proteins selected from the group consisting of: PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, and PDZD2 polypeptide in the sample;
wherein an increased level of one or more proteins is indicative of a poor prognosis.

45-56. (canceled)

57. A kit for the diagnosis of cancer comprising an antibody that specifically binds to one or more proteins selected from the group consisting of: PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, PDZD2, H1F0, PLEKHM1, SERPINB2, MX1, KRT6C, ISGF3G, IFI44, IFIT1, IFI44L, ADAMTS1, pTR7, DHRS2, IL8, CXCL2, MIRH1, ILIR2, ZCCHC2, UBE2E1, ZNF358, H1F0, KRT6C, KRT6A, ISGF3G, MX2, FLJ20035, ATP8A2, pTR7, TNC, SNHG7, A_{—}24_P707102, SERPINB2, NAV3, HIST1H2BM, LOC391566, EIF4EBP2, HISTH2BK, SCML1, and ANXA11, and instructions for use, or
A kit for determining the prognosis of a subject having cancer comprising an antibody that specifically binds to one or more proteins selected from the group consisting of: PLEKHM1, KRTAP3-1, MB2, DERP12, ZA31P, A_{—}24_P932355, PCSK9, RC1, JAK3, BC038245, HSPA2, SOST, METTL7A, NGEF, GPR30, GLRX, A_{—}23_P72014, L3MBTL, KCNMB4, GNAZ, AK096109, COL9A3, ARF3, HMOX1, CDH16, MX1, LY6D, IFI27, GLI1, IFITM1, ISG15, LOC730999, LOXL4, PSCA, TGFB1, IFI44L, S100P, HTRA3, CXCR7, OLFML2A, IFI6, KRT4, PRSS23, PDZD2, H1F0, PLEKHM1, SERPINB2, MX1, KRT6C, ISGF3G, IFI44, IFIT1, IFI44L, ADAMTS1, pTR7, DHRS2, IL8, CXCL2, MIRH1, IL1R2, ZCCHC2, UBE2E1, ZNF358, H1F0, KRT6C, KRT6A, ISGF3G, MX2, FLJ20035, ATP8A2, pTR7, TNC, SNHG7, A 24 P707102, SERPINB2, NAV3, HIST1H2BM, LOC391566, EIF4EBP2, HISTH2BK, SCML1, and ANXA11, and instructions for use.

58-60. (canceled)
